(19) United States
(12) Patent Application Publication
(10) Pub. No.: US 2014/0220660 A1
(43) Pub. Date: Aug. 7, 2014

(54) PROCESS FOR THE PRODUCTION OF FILAMENTOUS BACTERIOPHAGE

(71) Applicants: Jason Wright, San Francisco, CA (US); Marc Bradford, Macclesfield (GB); Frank Sugar, Athens, GA (US); Tim Davies, Llandysul (GB); Kevin Millsap, Watkinsville, GA (US)

(72) Inventors: Jason Wright, San Francisco, CA (US); Marc Bradford, Macclesfield (GB); Frank Sugar, Athens, GA (US); Tim Davies, Llandysul (GB); Kevin Millsap, Watkinsville, GA (US)

(21) Appl. No.: 14/234,500

(22) PCT Filed: Jul. 27, 2012
(86) PCT No.: PCT/US2012/048565

§ 371 Date: Feb. 11, 2014

(30) Foreign Application Priority Data
Jul. 27, 2011 (US) 61512169

(51) Int. Cl. C12N 7/00

(52) U.S. Cl. CPC C12N 7/00
USPC 435/236; 435/235.1

(57) ABSTRACT

The invention relates to culture conditions and methods that allow reproducible production of high titers of filamentous bacteriophage. Culture media comprising high titers of filamentous bacteriophage, as we methods of producing high titers of filamentous phage on a large scale are encompassed.

This application claims the benefit of priority of U.S. Provisional Patent Application No. 61/512,169, filed Jul. 27, 2011, which is incorporated by reference in its entirety herein.

The invention relates to culture medium having high concentrations of filamentous bacteriophage such as M13, as well as methods for producing the same.

Filamentous bacteriophage have recently been suggested to have commercial use as therapeutics (WO2002074243, WO2006083795, WO2007001302, WO2008011503), in nanotechnology applications (Naik R R et at (2002) Nat Mater 1(3):169-172; Flynn C E et al (2003) J Mater Chem 13(10):2414-2421), as biofilms to decrease metal corrosion (Zuo R, et al (2005) Appl Microbiol Biotechnol 68(4):505-509), and in biomining (Curtis S B et al (2009) Biotechnol Bioeng 102(2):644-650). In addition, filamentous bacteriophage are routinely used to create display libraries of random peptides and as sequencing vectors.

Filamentous bacteriophage M13, and related filamentous phage, have shown utility in animal models of protein misfolding disease, and therefore represent potential therapeutic class for protein misfolding diseases. See paragraphs 96-117 of United States patent publication US 2011/0142803, incorporated by reference herein in its entirety. In particular, it has been discovered that filamentous bacteriophage have the ability to prevent plaque aggregation, as well as to dissolve aggregates that have already formed in the brain. See, e.g., WO2006083795 and WO2010060073, incorporated by reference herein in its entirety.

Plaque forming diseases are characterized by neuronal degeneration and the presence of misfolded, aggregated proteins in the brain. These misformed and aggregated proteins vary in different diseases, but in most cases, they have a beta-pleated sheet structure that stains with Congo Red dye. Removal of plaques is expected to reduce, slow the progression of, or even to reverse the symptoms associated with a variety of diseases characterized by plaques in the brain.

Neurodegenerative diseases known to be associated with misfolded and/or misaggregated protein in the brain include Alzheimer's disease, Parkinson's disease, prion diseases, amyotrophic lateral sclerosis (ALS), spinocerebellar ataxia (SCA1), (SCA3), (SCA6), (SCA7), Huntington disease, entatorubral-pallidoluysian atrophy, spinal and bulbar muscular atrophy, hereditary cerebral amyloid angiopathy, familial amyloidosis, frontotemporal lobe dementia, British/Danish dementia, and familial encephalopathy. There is a great need to prevent and/or reduce plaque formation (i.e., misfolded and/or misaggregated proteins) in the brain to treat or reduce the symptoms or severity of these diseases.

Filamentous bacteriophage are a group of structurally related viruses that infect bacterial cells, and contain a circular single-stranded DNA genome. They do not kill their host during productive infection. Rasched and Oberer, Microbiol Rev (1986) 50:401-427. Filamentous phage belong to a class of phage known as Ff, comprised of strains M13, f1, and fd (Rasched and Oberer, Microbiol Rev (1986) 50:401-427). The nucleotide sequence of fd has been known since 1978. Beck et al., Nucleic Acids Research (1978) 5(12):4495-4503. The full sequence of M13 was published in 1980, van Wezenbeek et al., Gene (1980) 11:129-148. Phage f1 was sequenced by 1982. Hill and Petersen, J. Virol. (1982) 44(1):32-46. The f1 genome comprises 6407 nucleotides, one less than phage fd. It differs from the fd sequence by 186 nucleotides (including one nucleotide deletion), leading to 12 amino acid differences between the proteins of phages f1 and fd. The f1 sequence differs from that of M13 by 52 nucleotides, resulting in 5 amino acid differences between the corresponding proteins. Id. The DNA sequences of M13 and fd vary at 192 (3%) nucleotides, yet only 12 of these differences result in a change in the corresponding amino acid sequence (6.25%), van Wezenbeek et al., Gene (1980) 11:129-148.

Having evolved for prokaryotic infection, assembly, and replication, bacteriophage can neither replicate in, nor show natural tropism for, mammalian cells. This minimizes the chances of non-specific gene delivery when used as a therapeutic in mammalian cells. Thus, phage vectors are potentially much safer than viruses as they are less likely to generate a replication-competent entity in animal cells (Monaci et al., Curr Opin Mol Ther. (2001) April; 3(2):159-69).

Filamentous bacteriophage are currently produced in small batches, in shake flasks, for example. More recently, controlled fermentors have been used (Grieco et al., Bioprocess Biosyst Eng (2009) 32(6) 773-79). However, even in the descriptions of production using fermentors, there have been none showing that high concentrations of filamentous bacteriophage can be reproducibly produced, or that they can be produced on a large scale. Thus, there is a need in the art for reproducible large-scale production of filamentous bacteriophage of high concentration for use, for example, in treating neuronal diseases and disorders that are characterized by plaque formation.

The invention disclosed herein is based in part on the discovery of culture conditions and methods that allow reproducible production of high concentrations of filamentous bacteriophage such as M13. It is also based in part on the discovery that high concentrations of filamentous bacteriophage can be produced in large scale preparations. Methods of producing high concentrations of filamentous bacteriophage on a large scale are vital for the commercial preparation of therapeutic filamentous bacteriophage to be used in the treatment and prevention of neuronal diseases and disorders.

Embodiments of the invention include culture media comprising filamentous bacteriophage (e.g., M13) having a concentration of at least 4×10¹² phage per mL. The invention also provides a fermentor comprising a culture medium comprising filamentous bacteriophage at a concentration of at least 4×10¹² filamentous bacteriophage per milliliter (mL), wherein the fermentor has a volume of at least 50 mL. The culture media and fermentors of the invention may also comprise filamentous bacteriophage such as M13 having at least 1×10¹³ phage per mL, 1×10¹³ to 9×10¹³ phage per mL, 1×10¹³ to 1×10¹⁴ phage per mL, 1×10¹³ to 9×10¹⁴ phage per mL, or 1×10¹⁴ to 9×10¹⁴ phage per mL.

Another aspect of the invention provides methods for reliably and reproducibly producing filamentous bacteriophage (e.g., M13) in culture media having a concentration of at least 4×10¹² phage per mL or in some embodiments, of at least 1×10¹³-2×10¹³ phage per mL. The invention also encompasses recombinant filamentous bacteriophage and methods of producing recombinant filamentous bacteriophage.

Also provided are methods for reproducibly producing large scale cultures of filamentous bacteriophage, such as, for example, M13. Such embodiments of the invention include the following.

The invention provides a method of producing a culture medium comprising greater than 4×10¹² filamentous bacteriophage per mL, comprising:

a) providing in a fermentor a culture comprising E. coli of a strain that expresses an F pilus contacted with a liquid culture medium;

b) adding filamentous bacteriophage to the culture in the fermentor, wherein the addition occurs either during the provision of step (a), or after beginning incubation according to step (c);

c) incubating the culture continuously or discontinuously for a duration totaling at least 36 hours, during which:
(i) dissolved oxygen in the culture is maintained at a concentration at or above 20%;
(ii) pH in the culture is maintained at or above 6.5; and
(iii) the culture is maintained at a temperature ranging from 30° C.-39° C.;

d) providing a supplemental carbon source to the culture as a feed beginning at a time between 3 and 7 hours after initiating incubation; and

e) ending incubation after the concentration of filamentous bacteriophage in the culture reaches a concentration greater than 4×10¹² filamentous bacteriophage per mL.

The invention provides a method of producing a culture medium comprising greater than 4×10¹² filamentous bacteriophage per mL, comprising;

a) providing in a fermentor, a mixture comprising filamentous bacteriophage contacted with a liquid culture medium;

b) contacting E. coli of a strain that expresses an F pilus with the liquid culture medium to form a culture;

c) incubating the culture continuously or discontinuously for a duration totaling at least 36 hours, during which:
(i) dissolved oxygen in the culture is maintained at a concentration at or above 20%;
(ii) pH in the culture is maintained at or above 6.5; and
(iii) the culture is maintained at a temperature ranging from 30° C.-39° C.;

d) providing a supplemental carbon source to the culture as a feed beginning at a time between 3 and 7 hours after initiating incubation; and

e) ending incubation after the concentration of filamentous bacteriophage in the culture reaches a concentration greater than 4×10¹² filamentous bacteriophage per mL.

### BRIEF DESCRIPTION OF THE DRAWINGS

FIG. 1 shows growth of E. coli cultures infected at 22 h with M13 stock solution. Four replicate cultures are shown (“73”, “74”, “75”, and “76”). The production process was run at 5 L scale in four replicated fermentations. Defined medium was used with yeast extract and 10 g/L glucose, along with a feed containing 50% glucose, yeast extract and salts. A cell-free phage suspension was to be added at an OD₆₀₀ of 55±5 at a titer of 2.5×10⁸ phage/mL culture starting volume*OD. This addition level gave amounts of 8.75×10¹¹ phage/OD unit for the 5 L fermentor (starting volume 3500 mL), or a total of 4.81×10¹³ phage added per reactor at an OD of 55. Cultures were grown for at least 24 h after infection with continual feeding. The four 5 L fermentations (“73”, “74”, “75”, and “76”) displayed reproducible growth profiles.

FIG. 2 shows the glucose concentration in the four replicate cultures shown in FIG. 1. The glucose was initially consumed during the batch phase and was well controlled for the first 24 hours of feeding. Late in the feeding stage, possibly due to stress as more M13 is produced and the E. coli cellular machinery is taxed, glucose consumption is reduced and substrate accumulates in the medium.

FIG. 3 shows growth and M13 production (measured by ELISA) for one selected culture, X axis is in hours.

FIG. 4A-FIG. 4D show data obtained from an experiment that produced greater than 4×10¹² bacteriophage per mL of culture medium. FIG. 4A shows the agitation in rpms and the dissolved oxygen content in percent over the course of the experiment. FIG. 4B shows the temperature remaining constant at about 37 degrees Celsius throughout the experiment. FIG. 4C shows the pH and the amount of base added to control pH throughout the experiment. FIG. 4D shows the feed rate in percent and the feed total, in mL, throughout the experiment.

FIG. 5 depicts a typical standard curve for an ELISA assay to detect and quantitate titers of filamentous bacteriophage M13.

FIG. 6A-FIG. 6B show data obtained from a single fermentation run (Run 1 from Table 20) resulting in a high titer yield of M13. Exemplary Process 2 was followed. FIG. 6A shows the data regarding agitation, feed total (mL), and pH. FIG. 6B shows the data relating to the cumulative amount of base added during fermentation to control pH, OD₅₀₀, and dissolved oxygen (“DO2”).

FIG. 7A-FIG. 7B show data obtained from a single fermentation run (Run 2 from Table 20) resulting in a high titer yield of M13. Exemplary Process 2 was followed. FIG. 7A shows the data regarding agitation, feed total (mL), and pH. FIG. 7B shows the data relating to the cumulative amount of base added during fermentation to control pH, OD₆₀₀, and dissolved oxygen (“DO2”).

FIG. 8A-FIG. 8B show data obtained from a single fermentation run (Run 3 from Table 20) resulting in a high titer yield of M13. Exemplary Process 2 was followed. FIG. 8A shows the data regarding agitation, feed total (mL), and pH. FIG. 8B shows the data relating to the cumulative amount of base added during fermentation to control pH, OD₆₀₀, and dissolved oxygen (“DO2”).

FIG. 9A-FIG. 9B show data obtained from a single fermentation run (Run 4 from Table 20) resulting in a high titer yield of M13. Exemplary Process 2 was followed. FIG. 9A shows the data regarding agitation, feed total (mL), and pH. FIG. 9B shows the data relating to the cumulative amount of base added during fermentation to control pH, OD₅₀₀, and dissolved oxygen (“DO2”).

FIG. 10A-FIG. 10B show data obtained from a single fermentation run (Run 5 from Table 20) resulting in a high titer yield of filamentous bacteriophage. Exemplary Process 2 was followed. FIG. 10A shows the data regarding agitation, feed total (mL), and pH. FIG. 10B shows the data relating to the cumulative amount of base added during fermentation to control pH, OD₆₀₀, and dissolved oxygen (“DO2”).

FIG. 11A-FIG. 11B show data obtained from a single fermentation run (Run 1 from Table 21) resulting in a high titer yield of M13. Exemplary Process 3 was followed. FIG. 11A shows the data regarding agitation, feed total (mL), and pH. FIG. 11B shows the data relating to the cumulative amount of base added during fermentation to control pH, OD₆₀₀, and dissolved oxygen (“DO2”).

FIG. 12A-FIG. 12B show data obtained from a single fermentation run (Run 2 from Table 21) resulting in a high titer yield of M13. Exemplary Process 3 was followed. FIG. 12A shows the data regarding agitation, feed total (mL), and pH. FIG. 12B shows the data relating to the cumulative amount of base added during fermentation to control pH, OD₆₀₀, and dissolved oxygen (“DO2”).

FIG. 13A-FIG. 13B show data obtained from a single fermentation run (Run 3 from Table 21) resulting in a high titer yield of M13. Exemplary Process 3 was followed. FIG. 13A shows the data regarding agitation, feed total (mL), and pH. FIG. 13B shows the data relating to the cumulative amount of base added during fermentation to control pH, OD₆₀₀, and dissolved oxygen (“DO2”).

FIG. 14A-FIG. 14B show data obtained from a single fermentation run (Run 4 from Table 21) resulting in a high titer yield of M13. Exemplary Process 3 was followed. FIG. 14A shows the data regarding agitation, feed total (mL), and pH. FIG. 14B shows the data relating to the cumulative amount of base added during fermentation to control pH, OD₆₀₀, and dissolved oxygen (“DO2”).

FIG. 15 shows a plot of OD₆₀₀=versus time for the seven fermentation runs described in Example 12, for which Exemplary Process 4 was followed.

### DESCRIPTION OF EMBODIMENTS

### Definitions

Filamentous bacteriophage are a group of related viruses that infect gram negative bacteria, such as, e.g., E. coli. See, e.g., Rasched and Oberer, Microbiology Reviews (1986) December: 401-427. In the present application, filamentous bacteriophage may also be referred to as “bacteriophage,” or “phage.” Unless otherwise specified, the term “filamentous bacteriophage” includes both wild type filamentous bacteriophage and recombinant filamentous bacteriophage.

“Wild type filamentous bacteriophage” refers to filamentous bacteriophage that express only filamentous phage proteins and do not contain any heterologous nucleic acid sequences, e.g. non-phage sequences that have been added to the bacteriophage through genetic engineering or manipulation. One such wild type filamentous bacteriophage useful in the invention is M13. The term “M13” is used herein to denote a form of M13 phage that only expresses M13 proteins and does not contain any heterologous nucleic acid sequences, M13 proteins include those encoded by M13 genes I, II, III, IIIp, IV, V, VI, VII, VIII, VIIIp, IX and X. van Wezenbeek et al. Gene (1980) 11:129-148.

Suitable wild type filamentous bacteriophage useful in this invention include at least M13, f1, or fd. Although M13 was used in the Examples presented below, any closely related wild type filamentous bacteriophage is expected to behave and function similarly to M13. Closely related wild type filamentous bacteriophage refers to bacteriophage that share at least 85%, at least 90%, or at least 95% identity to the sequence of M13, f1, or fd at the nucleotide or amino acid level. In some embodiments, closely related filamentous bacteriophage refers to bacteriophage that share at least 95% identity to the DNA sequence of M13 (See, e.g., GenBank: V00604; Refseq: NC 003287).

“Recombinant filamentous bacteriophage” refers to filamentous bacteriophage that have been genetically engineered to express at least one non-filamentous phage protein and/or comprise least one heterologous nucleic acid sequence. For example, recombinant filamentous bacteriophage may be engineered to express a therapeutic protein, including, e.g., an antibody, an antigen, a peptide that inhibits or activates a receptor, a peptide composed of beta-breaker amino acids like proline, cyclic peptides made of alternating D and L residues that form nanotubes, and a metal binding peptide.

Dissolved oxygen may be referred to as “DO,” “DO2,” or DO₂” throughout.

The culture media of the invention may be produced in any desired volume by adjusting the processes set forth below as necessary and as would be readily understood by those of skill in the art. In some embodiments, the culture medium is produced in 5 L batches. In other embodiments, the culture medium is produced in 0.05, 0.1, 0.2, 0.5, 1, 2, 10, 20, 50, 100, 1,000, 2,000, 5,000, 10,000, 20,000, 40,000, 60,000, 80,000 or 100,000 L batches. Correspondingly, fermentors comprising culture medium with bacteriophage according to the invention may have a volume of at least 0.05 L (50 mL), e.g., 0.05, 0.1, 0.2, 0.5, 1, 2, 5, 10, 20, 50, 100, 1,000, 2,000, 5,000, 10,000, 20,000, 40,000, 60,000, 80,000 or 100,000 L. With respect to a fermentor, volume refers to an amount of culture medium that can be incubated in the fermentor. In each embodiment, the culture media comprise wild type filamentous bacteriophage or recombinant filamentous bacteriophage at a concentration of at least 4×10¹² phage/mL. In some embodiments, the culture media comprise filamentous bacteriophage or recombinant filamentous bacteriophage at a concentration of at least 1×10¹³ phage/mL. Thus, 5 L embodiments of the culture media comprise at least 2×10¹⁶ total phage or least 5×10¹⁶ total phage; 20 L embodiments of the culture media comprise at least 8×10¹⁶ total phage or at least 2×10¹⁷ total phage; 100 L embodiments comprise at least 4×10¹⁷ total phage or at least 1×10¹⁸ total phage; 1,000 L embodiments comprise at least 4×10¹⁸ or at least 1×10¹⁹ total phage; and 100,000 L embodiments comprise at least 4×10²⁰ total phage or at least 1×10²¹ total phage.

“Culture media” or “culture medium” as used herein is the media in which the filamentous bacteriophage grow, prior to any concentration or purification steps. Culture medium may also comprise E. coli, such as E. coli of a strain that expresses an F pilus.

“Maintain” as used herein means to keep a parameter at an indicated specification or to adjust the parameter back quickly (e.g., within 5 minutes, 1 minute, 30 seconds, or less, or as soon as possible) upon detection of a deviation.

A “monosaccharide” (commonly known as a simple sugar) is a polyhydroxy alcohol containing either an aldehyde or a ketone group, which may exist as or be in equilibrium with a cyclic hemiacetal form rather than an aldehyde or ketone form. Exemplary monosaccharides include, but are not limited to, mannose, glucose, galactose, xylose, arabinose, ribose and fructose. Many monosaccharides are chiral and have enantiomers (traditionally designated L and D forms). As used herein, references to monosaccharides, whether generic or specific, are to the form(s) metabolizable by E. coli (e.g., D-glucose), unless the context indicates otherwise.

An “oligosaccharide” is a linear or branched carbohydrate that consists of from two to ten monosaccharide units joined by means of glycosidic bonds. Oligosaccharides include, but are not limited to disaccharides (a “disaccharide” being an oligosaccharide consisting of two monosaccharide units joined by means of a glycosidic bond) such as sucrose, trehalose, lactose and maltose. Unless the context indicates otherwise, the monosaccharide units making up an oligosaccharide are of the enantiomeric form(s) metabolizable by E. coli (e.g., D-glucose).

A “sugar alcohol” is an alcohol derivative of a mono- or an oligosaccharide which is generally formed by reduction of the aldehyde or ketone moiety on the mono- or oligosaccharide. Exemplary sugar alcohols include, but are not limited to, mannitol, sorbitol, arabitol, inositol, galactitol, erythritol, xylitol, and threitol. Unless the context indicates otherwise, sugar alcohols derived from monosaccharides are derived from the monosaccharide enantiomeric form(s) metabolizable by E. coli (e.g., D-glucose), and sugar alcohols derived from oligosaccharides are derived from oligosaccharides made up of monosaccharide units of the enantiomeric form(s) metabolizable by E. coli (e.g., D-glucose).

### Features and Embodiments of Fermentors and Processes for Reproducibly Producing High Concentrations of Filamentous Bacteriophage

Fermentors and processes for reproducibly producing high concentrations of filamentous bacteriophage according to the disclosure can comprise (a) providing in a fermentor a culture comprising E. coli of a strain that expresses an F pilus contacted with a liquid culture medium and adding filamentous bacteriophage to the culture in the fermentor, wherein the addition occurs either during the provision of step (a), or after beginning incubation as discussed below. Alternatively, fermentors and processes for reproducibly producing high concentrations of filamentous bacteriophage according to the disclosure can comprise providing in a fermentor, a mixture comprising filamentous bacteriophage contacted with a liquid culture medium and contacting E. coli of a strain that expresses an F pilus with the liquid culture medium to form a culture. Thus, infecting the bacteria with filamentous bacteriophage can occur either at the time of introduction into the fermentor or at a later time. The host bacteria strain can be, for example, JM109 (available from the ATCC; No. 53323), or JM107 (available from the ATCC; No. 47014). Types of filamentous bacteriophage that can be used are discussed above. The fermentor can comprise, for example, a tank made of stainless steel.

Processes according to the invention generally comprise incubating the culture continuously or discontinuously while maintaining conditions as discussed below for a duration totaling at least 36 hours. Longer durations of continuous or discontinuous incubation are also possible (see below). As noted above, in some embodiments, filamentous bacteriophage are added after beginning this incubation. The incubation may be discontinuous, for example, in that there may be brief deviations of culture conditions (e.g., pH or DO may go outside a range before being adjusted, as discussed in the definition section above with respect to the term “maintain”) and also in that procedures such as agitation and/or feed may be paused, e.g., at the time of addition of filamentous bacteriophage. Pauses can be of a set duration, or resumption of the paused procedure can be triggered by occurrence of a condition, as discussed in greater detail below. Such brief deviations and pauses generally do not substantially affect bacterial growth.

The culture conditions in the fermentor comprise providing a culture medium. Culture media such as modified Riesenberg media (see Examples) may be used. The culture medium is understood to comprise a carbon source, such as at least one monosaccharide, oligosaccharide (which may be a disaccharide), or sugar alcohol (which may be a monosugar alcohol). In some embodiments, the carbon source comprises at least one of the monosaccharide, oligosaccharide (which may be a disaccharide), or sugar alcohols listed in the definitions section. Exemplary ranges of initial carbon source concentrations are 8-12 g/L for oligo- or monosaccharides, e.g., glucose, and 8-40 g/L for sugar alcohols, e.g., glycerol. Lower ranges are possible, but it may become advisable to add additional carbon source (as discussed below) at an earlier time. Accumulation of acetate above 5 g/L can have inhibitory effects on E. coli growth. This can result from the presence of a high concentration of a carbon source, such as glucose, which can be metabolized to acetate through an anaerobic pathway. Accordingly, in some embodiments, the combined concentration in the culture medium of the initial carbon source which has not yet been metabolized and the additional carbon source which has been added but not yet metabolized does not exceed 40 g/L during the fermentation, or does not exceed 12 g/L during the fermentation. In some embodiments, a sugar alcohol is provided as carbon source and the combined concentration in the culture medium of the initial sugar alcohol which has not yet been metabolized and the additional sugar alcohol which has been added but not yet metabolized does not exceed 40 g/L during the fermentation. In some embodiments, an oligosaccharide is provided as carbon source and the combined concentration in the culture medium of the initial oligosaccharide which has not yet been metabolized and the additional oligosaccharide which has been added but not yet metabolized does not exceed 12 g/L during the fermentation. In some embodiments, glycerol is provided as carbon source and the combined concentration in the culture medium of the initial glycerol which has not yet been metabolized and the additional glycerol which has been added but not yet metabolized does not exceed 40 g/L during the fermentation. In some embodiments, glucose is provided as carbon source and the combined concentration in the culture medium of the initial glucose which has not yet been metabolized and the additional glucose which has been added but not yet metabolized does not exceed 12 g/L during the fermentation.

Additional carbon source can be added during the fermentation process. Additional carbon source (such as glucose or glycerol) can be provided as a feed when the initial carbon source is almost depleted, usually at 3-7 hours after start of fermentation. In some embodiments, the feed is initiated at a time ranging from 3.5 to 7 hours, 4 to 7 hours, from 4 to 6.5 hours, from 4 to 6 hours, from 4.5 to 6 hours, from 5 to 6 hours, from 5.5 to 6 hours, from 4 to 5.5 hours, from 4 to 5 hours, from 4.5 to 5.5 hours, or from 4.5 to 5 hours. The additional carbon source can be provided, for example, at a rate between 0.5-1.6 g/L/h, or alternatively 0.5-3.2 g/L/h (“the feed rate”). Initiation of feed at a time earlier than 3.5 hours is also possible. The additional carbon source may be accompanied by Mg²⁺, yeast extract and a buffering solution.

A base such as ammonium hydroxide can be added during fermentation to prevent the culture from becoming overly acidic. For example, base can be added to maintain pH above a level ranging from 6.0 to 7.5, e.g., above 6.0, 6.1, 6.2, 6.3, 6.4, 6.5, 6.6, 6.7, 6.8, 6.9, 7.0, 7.1, 7.2, 7.3, 7.4, or 7.5.

Dissolved oxygen (DO) is maintained during at least part of the fermentation. Possible manners of maintaining DO include air flow, agitation, and oxygen-supplemented air flow, discussed in more detail below. In some embodiments, DO is maintained in the fermentation culture medium at a concentration of at least 20% to 40%, e.g., at least 20%, at least 30%, at least 35%, or at least 40%. Maintenance of DO at or above a higher value is also possible. All percentage values of DO recited herein are expressed relative to the air saturation level, i.e., 100% DO indicates that the medium is fully saturated with air (of which about 21% is oxygen by volume). DO concentration can be maintained in any of a variety of ways, for example, by providing air flow, agitating the culture, supplementing the culture with pure oxygen, and/or pressurizing the fermentor. An exemplary range for the air flow is 0.5-2 volumes air/volume liquid/minute (vvm). These approaches can be combined; for example, air flow and agitation can be used together. In some embodiments, the DO level is controlled by a cascaded control loop, wherein the primary response to a change in DO is to alter the agitation rate (between 200 and 1000 rpms), and the secondary response to a change in DO is to supplement the air flow line with oxygen. In another exemplary embodiment, the air flow rate is adjusted as needed depending on the DO. In another exemplary embodiment, altering the pressure in the fermentor is used to keep the level of DO within the desired range. As the maintenance of DO in the range of 20% to 40%

In some embodiments, the methods comprise transferring host bacteria that were grown in shake flasks into the fermentor. However, the method by which the host bacteria provided for fermentation are prepared is not critical and can be chosen from, for example, bacteria grown in liquid media (in which aeration can be provided by, for example, rolling, shaking, or bubbling air or oxygen through the media) or any other suitable method for growing bacteria. In some embodiments, bacteria are provided which have been cultured in at least two stages prior to fermentation, with the culture volume increasing from stage to stage. The volumes of these cultures is not critical, but for a 5 L fermentation scale, an exemplary range for the first culture stage is 1-30 mL, and an exemplary range for the second culture stage is 20-500 mL, wherein the second volume is greater than the first volume. It is also possible to prepare host bacteria for a fermentation according to the disclosure by a preliminary fermentation step, or by growth in a chemostat. It is not necessary to use the same media to grow bacteria prior to the fermentation step as is used during fermentation. In some embodiments, prior to being contacted with the liquid culture medium the E. coli are: (i) grown for at least two doublings in a separate liquid culture; and (ii) not frozen after the at least two doublings. In some embodiments, prior to being contacted with the liquid culture medium the E. coli are (i) grown for at least two doublings in a first liquid culture in a first vessel; (ii) grown for at least two doublings in a second liquid culture in a second vessel, and (iii) not frozen after the at least two doublings in the first vessel.

Phage for use in methods according to the disclosure can be prepared by standard methods, e.g., obtaining the phage from an infected shake flask culture of host bacteria. Phage obtained from a previous fermentation can also be used.

The precise timing of phage addition is not critical. For example, phage can be added at the time of transferring the host bacteria into the fermentor, as described below with respect to Exemplary Process 3.

Alternatively, phage can be added later, during fermentation. For example, the infection step can be performed when the OD of the culture in the fermentor is in the range of 35 to 75, 40 to 70, 45 to 70, 45 to 65, 45 to 60, 45 to 55, 50 to 75, 50 to 70, or 50 to 65.

In a further variation, phage can be added to the fermentor prior to addition of bacteria. Thus, in some embodiments, methods according to the invention for producing a culture medium comprising greater than 4×10′² filamentous bacteriophage per mL can comprise:

a) providing in a fermentor, a mixture comprising filamentous bacteriophage contacted with a liquid culture medium;

b) contacting E. coli of a strain that expresses an F pilus with the liquid culture medium to form a culture;

c) incubating the culture continuously or discontinuously for a duration totaling at least 36 hours, during which:
(i) dissolved oxygen in the culture is maintained at a concentration at or above 20%;
(ii) pH in the culture is maintained at or above 6.5; and
(iii) the culture is maintained at a temperature ranging from 30° C.-39° C.;

d) providing a supplemental carbon source to the culture as a feed beginning at a time between 3 and 7 hours after initiating incubation; and

e) ending incubation after the concentration of filamentous bacteriophage in the culture reaches a concentration greater than 4×10¹² filamentous bacteriophage per mL.

The amount of phage that is added is generally expressed as phage per OD₆₀₀ per mL of culture starting volume, such that if, for example, 1×10⁶ phage/OD₆₀₀/mL were to be added to a 1 L culture with OD₆₀₀=1, 10⁹ phage would be added; if 1×10⁶ phage/OD600/mL were to be added to a 5 L culture with OD₆₀₀=20, 10¹¹ phage would be added; and so on. In some embodiments, the amount of phage added ranges from 5×10⁴ to 5×10⁸ phage/OD₆₀₀/L, 1×10⁵ to 1×10¹⁹ phage/OD₆₀₀/mL, 5×10⁴ to 1×10⁹ phage/OD₆₀₀/mL, 1×10⁵ to 5×10³ phage/OD₆₀₀/mL, 5×10⁴ to 5×10⁷ phage/OD₆₀₀/mL, 5×10⁴ to 2×10⁷ phage/OD₆₀₀/mL, 1×10⁵ to 5×10⁷ phage/OD₆₀₀/mL, 5×10⁴ to 1×10⁷ phage/OD₆₀₀/mL, 1×10⁵ to 1×10⁷ phage/OD₆₀₀/mL, 5×10⁴ to 5×10⁶ phage/OD₆₀₀/mL, 1×10⁵ to 5×10⁶ phage/OD₆₀₀/mL, 1×10⁵ to 2.5×10′ phage/OD₆₀₀/mL, 5×10⁴ to 2.5×10⁶ phage/OD₆₀₀/mL, 1×10⁷ to 1×10⁹ phage/OD₆₀₀/mL, 2.5×10⁷ to 1×10⁹ phage/OD₆₀₀/mL, 2.5×10⁷ to 5×10⁸ phage/OD₆₀₀/mL, 5×10⁷ to 1×10⁹ phage/OD₆₀₀/mL, 5×10⁷ to 5×10⁸ phage/OD₆₅₀/mL, or 1×10³ to 5×10⁸ phage/OD₆₀₀/mL.

The phage may be provided as freshly grown phage or from a thawed freezer stock. The examples that follow provide an example of a procedure that can be used to make a freezer stock of phage. Phage can be obtained from infected bacteria in a shake flask, fermentor, or other culture vessel.

The agitation and/or feed rates may be reduced or suspended during and/or following the addition of phage, as discussed in detail with respect to exemplary processes 1 and 2 below. If a period of reduced or suspended feed and/or agitation is used, its duration can be, for example, 10-90, 20-75, or 25-45 minutes. Alternatively, resumption of feed can be triggered by DO level rather than a set time period; for example, feed can be resumed when DO increases above 20%. This period can include a phase during which agitation is gradually ramped up. If both agitation and feed are reduced or suspended, they may or may not be reduced or suspended for identical durations. Afterward, normal feed and agitation are resumed; agitation subject to cascade control for DO maintenance as discussed above qualifies as normal agitation.

As is apparent from the above discussion, there can be times in the fermentation process during which the maintaining of parameters (e.g., DO level) is paused. However, processes according to the invention will generally comprise incubating the culture for a period or periods of incubation having a collective duration totaling at least 36 hours during which incubation parameters such as DO level, pH, and temperature are maintained; in some embodiments, the collective duration is at least 37, 38, 39, 40, 41, 42, 43, 44, 45, 46, 47, or 48 hours.

Processes according to the invention generally comprise ending incubation after the concentration of filamentous bacteriophage in the culture reaches a concentration greater than 4×10¹² filamentous bacteriophage per mL. Ending incubation can mean removing filamentous bacteriophage from the fermentor and/or ceasing maintenance of fermentation parameters (e.g., pH, DO, temperature, feed rate). Thus, for example, after the concentration of filamentous bacteriophage in the culture reaches a concentration greater than 4×10¹² filamentous bacteriophage per mL, ceasing feed, ceasing agitation, or deactivating cascade control of DO level or a thermostat responsible for temperature maintenance would constitute ending the incubation. In some embodiments, the ending of the incubation occurs after the filamentous bacteriophage in the culture reaches a concentration greater than at least 1×10¹³ phage per mL, 1×10¹³ to 9×10¹³ phage per mL, 1×10¹³ to 1×10¹⁴ phage per mL, 1×10¹³ to 9×10¹⁴ phage per mL, or 1×10¹⁴ to 9×10¹⁴ phage per mL. In some embodiments, the incubation is ended when the culture comprises at least a certain number of filamentous bacteriophage, such as least 2×10¹⁶, 5×10¹⁶, 8×10¹⁶, 2×10¹⁷, 4×10¹⁷, 1×10¹⁸, 4×10¹⁸, 1×10¹⁹, 4×10²⁰, or 1×10²¹ total phage.

### Exemplary Process 1

The steps for reproducibly producing high concentrations of filamentous bacteriophage such as M13 may comprise the following.

A host bacterial E. coli strain, such as, for example, JM109, JM107 or other strains of E. coli expressing an F pilus, are grown in a shake flask in an incubated shaker at 37° C. and 250 rpm until the culture reaches an OD₆₀₀ between 1 and 20. An OD₆₀₀ between 1 and 4 is typically achieved between 20 and 24 hours of growth when grown in Minimal media. The media may be any media known to support growth of E. coli, such as, for example, Minimal media, Luria Bertani (LB) and Terrific Broth (TB).

After the E. coli culture has reached an OD₆₀₀ between 1 and 20, the E. coli culture is transferred to a fermentor by diluting approximately 1:40 into a starting volume of modified Riesenberg media (see, Riesenberg et al., Journal of Biotechnology (1991) 20:17-28 and the example section for modifications). For example, 100 mL of E. coli culture is transferred to a fermentor containing 4 L of modified Riesenberg media. Scaling up or down follows this ratio.

The conditions or parameters for growth of the E. coli culture and the infected E. coli culture in a 5 L fermentor (“fermentation parameters”) are kept constant as follows. Scaling up or down to allow for a smaller or larger scale fermentation follows these guidelines:
a. agitation of between 200 and 1,000 rpm, and in some embodiments between 300 and 600 rpm;
b. an initial energy source, such as, for example, glucose or glycerol, and optionally yeast extract, a buffering solution, trace elements, and thiamine, wherein the media in the fermentor is a modified Riesenberg media (see Examples), and has a starting concentration of glucose of between 8 and 12 grams per liter (L), or glycerol between 8 and 40 grams per L. When this initial energy source is almost depleted (about 5-7 hours after start of fermentation), additional glucose or glycerol is provided at a rate between 0.5-1.6 g/L/h, or alternatively 0.5-3.2 g/L/h (“the feed rate”). The additional glucose or glycerol may be accompanied by Mg²⁺, yeast extract and a buffering solution;
c. dissolved oxygen (“DO”) of between 20% and 40% including, for example, 20, 25, 30, 35, or 40%, controlled by a cascaded control loop, wherein the primary response to a change in DO is to alter the agitation rate (between 200 and 1000 rpms), and the secondary response to a change in DO is to supplement the air flow line with pure oxygen. In another exemplary embodiment, the air flow rate discussed in step (d) is not kept constant, but is adjusted as needed depending on the DO. In another exemplary embodiment, altering the tank pressure is used as a supplemental DO control strategy (e.g., when a stainless steel system is utilized);
d. an air flow rate between 0.5-2.0 volume/volume/minute (vvm);
e. a pH of not less than 6.5; and
f. temperature between 30° C. and 39° C. including, for example, 30, 31, 32, 33, 32, 35, 36, 37, 38, or 39° C.

If needed, an antifoam reagent is added during any stage of fermentation.

The dissolved oxygen is kept constant between 20% and 40% by continually measuring the dissolved oxygen content, and adjusting the amount of agitation accordingly. An automated feedback loop can be used for monitoring DO and adjusting agitation. For example, if the dissolved oxygen threatens to fall below 20%, agitation may be increased. If the dissolved oxygen threatens to rise past 40%, agitation may be decreased. If agitation cannot maintain the dissolved oxygen content between 20 and 40%, pure oxygen may be added. Oxygen may be supplemented into the 0.5-2.0 vvm air flow by the opening of a valve (controlled by the digital control unit as part of the cascade control loop). Alternatively, the dissolved oxygen percentage may also be adjusted by placing the fermentation tank under pressure.

If the pH falls below 6.5, base is added.

The feed rate is adjusted between about 0.5-1.6 g/L/h, or alternatively 0.5-3.2 g/L/h, so that glucose (or glycerol) does not accumulate in the culture. Accumulated glucose of greater than 5 g/L can result in unwanted acetate accumulation and a reduction in the growth of bacterial cells.

Supplemental glucose or glycerol is typically added at between 3.25 and 7.25 hours after transfer to the fermentor, including, for example, 3.25, 3.5, 4.0, 4.5, 5.0, 5.5, 6.0, 6.25, 6.5, 7.0, or 7.25 hours after transfer to the fermentor. Glucose or glycerol is provided between 0.5 and 1.6 g/L/h, including, for example, 0.5, 0.6, 0.7, 0.8, 0.9, 1.0, 1.1, 1.2, 1.3, 1.4, 1.5 and 1.6 g/L/h, or alternatively 0.5-3.2 g/L/h.

Once the E. coli culture in the fermentor reaches an OD₆₀₀ between 50 and 70, the feed is stopped. Once a dissolved oxygen spike of greater than about 40% is noted, the agitation is stopped. Air flow is maintained, and the E. coli culture is infected with between 2.0×10⁸ and 3.0×10⁸ filamentous bacteriophage (e.g., M13) per milliliter (mL) of culture starting volume per unit OD₆₀₀. The filamentous bacteriophage may be added neat (i.e., without dilution) or diluted in PBS. A pipette, syringe or serological pipette may be used, for example. M13 may be also added through an addition bottle, bag or other vessel delivered by gravity, pressure or using a pump.

Following the infection with filamentous bacteriophage such as M13, the fermentor is incubated with no agitation for 20 to 40 minutes, including, for example, 20, 25, 30, 35, or 40 minutes. After the rest period, agitation is restarted and the fermentation parameters noted above are resumed.

The feed is resumed once the dissolved oxygen in the infected culture reaches about 20%. The feed comprises glucose or glycerol between 0.5 and 1.6 g/L/h, including, for example, 0.5, 0.6, 0.7, 0.8, 0.9, 1.0, 1.1, 1.2, 1.3, 1.4, 1.5 and 1.6 g/L/h, or alternatively 0.5-3.2 g/L/h.

The filamentous bacteriophage are harvested between 40 and 48 hours after inoculation of filamentous bacteriophage (e.g., M13) into the fermentor, or when the concentration of filamentous bacteriophage is at least 4×10¹² filamentous bacteriophage per milliliter (mL).

The yield of filamentous bacteriophage may be at least 1×10¹³ to 9×10¹³ phage per mL, 1×10¹³ to 1×10¹⁴ phage per mL, or 1×10¹⁴ to 9×10¹⁴ phage per mL.

In some embodiments, methods for producing a culture of filamentous bacteriophage having a concentration of at least 4×10¹² filamentous bacteriophage per mL according to exemplary process 1 comprise the steps of:
a) growing an E. coli culture until the culture reaches and OD₆₀₀ between 1 and 20, wherein the E. coli express an F pilus;
b) diluting the E. coli culture 1:40 in a fermentor;
c) maintaining the temperature of the fermentor between 30° C. and 39° C., the dissolved oxygen content between 20% and 40%, the pH at or above 6.5, the air flow between 0.5 and 2.0 volume per volume per minute (vvm), and the agitation between 300 and 1200 rpms (e.g., 300-600 rpms, 600-1200 rpms, or 1000-1200 rpms; higher rpms may be appropriate for smaller fermentors, and vice versa);
d) adding glucose at the start of the fermentation to a concentration of between 3 and 12 grams per liter and then diluting the E. coli culture into the fermentor, followed by the initiation of the feed between 4 and 7 hours at a rate between 0.5 and 1.6 grams per liter per hour;
e) ceasing the addition of glucose once the E. coli culture has reached an OD₆₀₀ between 50 and 60, and infecting the E. coli culture with between 2.0×10⁸ and 3.0×10⁸ filamentous bacteriophage per mL of the E. coli culture's starting volume per unit OD₆₀₀;
f) ceasing the agitation for 20 to 40 minutes after the infection with bacteriophage;
g) resuming the addition of glucose at a rate of about 0.5 and 1.6 grams per liter per hour; an
h) harvesting the filamentous bacteriophage 40-48 hours after the start of step (a) when the bacteriophage have a titer of at least 4×10¹² bacteriophage per mL.

In some embodiments of such methods, the OD600 of step a) is achieved after between 20 and 24 hours.

### Exemplary Process 2

A second process, which has a two-stage seed process, comprises at least the following steps.

A bacterial E. coli strain, such as, for example, JM109, JM107 or other strains of E. coli expressing an F pilus, are grown in a shake flask in an incubated shaker at 37° C. and 250 rpm for 6 to 30 hours, including, for example, 6, 7, 8, 9, 10, 11, 12, 13, 14, 15, 16, 17, 18, 19, 20, 21, 22, 23, 24, 25, 26, 27, 28, 29, or 30 hours. The media may be any media known to support growth of E. coli, such as, for example, Minimal media, Luria Bertani (LB) and Terrific Broth (TB).

After 6 to 30 hours, typically 20 to 24 hours, a volume of E. coli culture from the first shake flask is transferred into a second shake flask. Typically, the volume of E. coli culture to be transferred is between 0.5 and 20% of the volume of media to be transferred into, assuming the OD₆₀₀ of the E. coli culture is between 0.5 and 10 units. For example, 2.5-100 mL of E. coli culture may be transferred into a second shake flask containing 500 mL of media (assuming an OD₆₀₀ between 0.5 and 10 units). The media in the first and second shake flask may be any media known to support growth of E. coli, such as, for example, Minimal media, Luria Bertani (LB) and Terrific Broth (TB).

In the event a fermentor or other means to generate a high cell density culture is used instead of the shake flask for the first pie-culture and assuming the OD₆₀₀ of the E. coli culture is between 0.5 and 200 units, the volume to be transferred may be between 0.01 and 20% of the volume of media to be transferred into.

The second shake flask is grown for about 6 to 30 hours, including, for example, 6, 7, 8, 9, 10, 11, 12, 13, 14, 15, 16, 17, 18, 19, 20, 21, 22, 23, 24, 25, 26, 27, 28, 29, or 30 hours.

After 6 to 30 hours, a volume of E. coli culture from the second shake flask is transferred into a fermentor. Typically, the volume of E. coli culture to be transferred is between 0.5 and 20% of the volume of media to be transferred into, assuming the OD₆₀₀ of the E. coli culture is between 0.5 and 10 units. For example, 2.5-100 mL of E. coli culture may be transferred into a fermentor containing 500 mL of media (assuming an OD₆₀₀ between 0.5 and 10 units). The fermentor comprises modified Riesenberg media (see Examples), or media with similar ingredients.

In the event a fermentor or other means to generate a high cell density culture is used instead of a shake flask for the first or second pre-culture and assuming the OD₆₀₀ of the E. coli culture is between 0.5 and 200 units, the volume to be transferred would be between 0.01 and 20% of the volume of media to be transferred.

The conditions or parameters for growth of the E. coli culture and the infected E. coli culture in a 5 L fermentor (“fermentation parameters”) are kept constant as follows. Scaling up or down to allow for a smaller or larger scale fermentation follows these guidelines:
a. agitation of between 200 and 1,000 rpm, and in some embodiments between 300 and 600 rpm;
b. an energy source, such as, for example, glucose or glycerol, and optionally yeast extract, a buffering solution, trace elements, and thiamine. The media in the fermentor has a starting concentration of glucose or glycerol of between 3 and 7 grams per liter (L). When this energy source is almost depleted (about 3.5-7 hours after start of fermentation), additional glucose or glycerol is provided at a rate between 0.5-1.6 g/L/h, or alternatively 0.5-3.2 g/L/h (“the feed rate”). The additional glucose or glycerol may be accompanied by Mg²⁺, yeast extract and a buffering solution;
c. dissolved oxygen (“DO”) of between 20% and 40% including, for example, 20, 25, 30, 35, or 40%, controlled by a cascaded control loop, wherein the primary response to a change in DO is to alter the agitation rate (between 200 and 1000 rpms), and the secondary response to a change in DO is to supplement the air flow line with pure oxygen. In another exemplary embodiment, the air flow rate discussed in step (d) is not kept constant, but is adjusted as needed depending on the DO. In another exemplary embodiment, altering the tank pressure is used as a supplemental DO control strategy (e.g., when a stainless steel system is utilized);
d. an air flow rate of 0.5-2.0 volume/volume/minute (vvm);
e. a pH of not less than 6.5; and
f. temperature between 30° C. and 39° C. including, for example, 30, 31, 32, 33, 32, 35, 36, 37, 38, or 39° C.

The dissolved oxygen is kept constant between 20% and 40% by continually measuring the dissolved oxygen content, and adjusting the amount of agitation accordingly. An automated feedback loop can be used for monitoring DO and adjusting agitation. For example, if the dissolved oxygen threatens to fall below 20%, agitation may be increased. If the dissolved oxygen threatens to rise past 40%, agitation may be decreased. If agitation cannot maintain the dissolved oxygen content between 20 and 40%, pure oxygen may be added. Oxygen may be supplemented into the 0.5-2.0 vvm air flow by the opening of a valve (controlled by the digital control unit as part of the cascade control loop). Alternatively, the dissolved oxygen percentage may also be adjusted by placing the fermentation tank under pressure.

If the pH falls below 6.5, base is added.

A glucose or glycerol feed is initiated at between 3.5 and 7 hours after transfer to the fermentor, including, for example, 3.5, 4.0, 4.5, 5.0, 5.5, 6.0, 6.5 or 7 hours. Glucose or glycerol is provided between 0.5 and 1.6 g/L/h, including, for example, 0.5, 0.6, 0.7, 0.8, 0.9, 1.0, 1.1, 1.2, 1.3, 1.4, 1.5 and 1.6 g/L/h, or alternatively 0.5-3.2 g/L/h.

Once the E. coli culture in the fermentor reaches and OD₆₀₀ between 45 and 55, the E. coli culture is infected with between 2.0×10⁸ and 3.0×10⁸ filamentous bacteriophage, such as M13, per milliliter (mL) of culture starting volume per unit OD₆₀₀. The filamentous bacteriophage (e.g., M13) are typically diluted in PBS, for example, 50 mL of PBS for a 5 L final volume. The agitation is reduced to 100 rpm while pumping the bacteriophage into the fermentor at between 8 and 12 mL per minute over a 3 to 7 minute period. The air flow is maintained at 0.5-2.0 vvm and feed is continued as per the “fermentation parameters” throughout.

A pipette, syringe, or serological pipette may be used to inoculate the E. coli culture. Alternatively, the filamentous bacteriophage may be pumped in, transferred by gravity, or transferred by other means, from a suitable container or bag through an addition port.

After the bacteriophage have been added, the agitation is continued for 1 to 3 minutes at about 100 rpm. Agitation is then stopped, leaving aeration and feed on, for about 20 to 40 minutes. Agitation is then resumed and ramped from about 200 to about 500 rpm over 10 to 40 minutes. After this step, DO control is resumed per the fermentation parameters.

The filamentous bacteriophage are harvested between 40 and 48 hours after start of the E. coli in the shake flask, or 20 to 24 hours after inoculation of filamentous bacteriophage into the fermentor or when the concentration of filamentous bacteriophage is at least 4×10¹² filamentous bacteriophage per milliliter (mL).

The yield of filamentous bacteriophage may be at least 1×10¹³ to 9×10¹³ phage per mL, or 1×10¹⁴ to 9×10¹⁴ phage per mL.

Foaming may be controlled by bolus additions of antifoam, such as, for example, 20% Hydrite 3721 antifoam, at approximately 0 hrs, 4.5 hrs, 18 hrs, 24 hrs, 30 hrs, and 40 hrs, as needed. Antifoam may be added via syringe and needle through the septum port or pumped in through an addition bottle or other suitable reservoir.

In some embodiments, methods for producing a culture of filamentous bacteriophage having a concentration of at least 4×10¹² filamentous bacteriophage per mL according to exemplary process 2 comprise the steps of:
a) growing an E. coli culture in a first shake flask for 20 to 28 hours, wherein the E. coli express an F pilus;
b) transferring a volume of E. coli culture from the first flask into a second shake flask, wherein the volume to be transferred is between 0.5 and 20% of the volume of media to be transferred into;
c) growing the E. coli culture in the second shake flask for 20 to 28 hours;
d) transferring a volume of E. coli culture from the second flask into a fermentor, wherein the volume to be transferred is between 0.5 and 20% of the volume of media to be transferred into;
e) maintaining the temperature of the fermentor between 30° C. and 39° C., the dissolved oxygen content between 20% and 40%, the pH above 6.5, the air flow between 0.5 and 2.0 vvm, and the agitation between 300 and 1200 rpms (e.g., 300-600 rpms, 600-1200 rpms, or 1000-1200 rpms; higher rpms may be appropriate for smaller fermentors, and vice versa);
f) adding glucose at the start of the fermentation to a concentration of between 3 and 12 grams per liter and then diluting the E. coli culture into the fermentor, followed by the initiation of the feed between 4 and 7 hours at a rate between 0.5 and 1.6 grams per liter per hour;
g) infecting the E. coli culture in the fermentor with between 2.0×10⁸ and 3.0×10⁸ filamentous bacteriophage per mL of the culture's starting volume per unit 00600 once the E. coli culture has reached an OD600 between 45 and 55, wherein the agitation is reduced to 100 rpm during infection, and wherein the bacteriophage are added into the fermentor at between 8 and 12 milliters per minute over 3 to 7 minutes;
h) ceasing agitation for between 20 and 40 minutes;
i) resuming agitation at 200 rpms and increasing the agitation to 500 rpms over 10 to 40 minutes; and
j) harvesting the filamentous bacteriophage 40-48 hours after the start of step (a) when the bacteriophage have a titer of at least 4×10¹² bacteriophage per mL.

### Exemplary Process 3

A third process, which involves a two-stage seed process, comprises at least the following steps.

A bacterial E. coli strain, such as, for example, JM109, JM107 or other strains of E. coli expressing an F pilus, are grown in a shake flask in an incubated shaker at 37° C. and 250 rpm for 20 to 28 hours. For example, a 250 mL baffled Erlenmeyer flask with 100 mL of M9 Minimal medium is inoculated with 1 mL of glycerol stock E. coli, wherein each the stock E. coli contains 1 mL at 0.72 OD₆₀₀ units of E. coli strain JM109, JM107 or other F pilus expressing strain from a previously stored stock. The media may be any media known to support growth of E. coli, such as, for example, Minimal media, Luria Bertani (LB) and Terrific Broth (TB).

After growth for 20 to 28 hours, a volume of E. coli culture from the first shake flask is transferred into a second shake flask. Typically, the volume of E. coli culture to be transferred is between 0.5 and 20% of the volume of media to be transferred into, assuming the OD₆₀₀ of the E. coli culture is between 0.5 and 10 units. For example, 2.5-100 mL of E. coli culture may be transferred into a second shake flask containing 500 mL of media (assuming an OD₆₀₀ between 0.5 and 10 units). The media may be any media known to support growth of E. coli, such as, for example, Minimal media, Luria Bertani (LB) and Terrific Broth (TB).

In the event a fermentor or other means to generate a high cell density culture is used instead of the shake flask for the first pre-culture and assuming the OD₆₀₀ of the E. coli culture is between 0.5 and 200 units, typically the volume to be transferred would be between 0.01 and 20% of the volume of media to be transferred.

The second shake flask is grown for about 6 to 30 hours, including, for example, 6, 7, 8, 9, 10, 11, 12, 13, 14, 15, 16, 17, 18, 19, 20, 21, 22, 23, 24, 25, 26, 27, 28, 29, or 30 hours.

After 6 to 30 hours, a volume of E. coli culture from the second shake flask is transferred into a fermentor comprising modified Riesenberg or similar media (see Examples). Typically, the volume of E. coli culture to be transferred is between 0.5 and 20% of the volume of media to be transferred into, assuming the OD₆₀₀ of the E. coli culture is between 0.5 and 10 units. For example, 2.5-100 mL of E. coli culture may be transferred into a fermentor containing 500 mL of media (assuming an OD₆₀₀ between 0.5 and 10 units).

In the event a fermentor or other means to generate a high cell density culture is used for the second pre-culture and assuming the OD₆₀₀ of the E. coli culture is between 0.5 and 200 units, typically the volume to be transferred would be between 0.01 and 20% of the volume of media to be transferred.

The fermentor is immediately infected with filamentous bacteriophage such as M13. This may be termed “infection at time zero.” Infection at time zero is in contrast to processes 1 and 2, where the culture is allowed to reach a certain OD₆₀₀ in the fermentor before infection with filamentous bacteriophage.

The E. coli culture is infected with between 1.0 and 2.0×10¹³ total filamentous phage (or approximately 3.0 to 4.0×10¹² phage per L). M13 is encompassed. For example, 50 μL of M13 from a stock concentrated at 2.8×10¹⁴ page per mL.

The conditions or parameters for growth of the E. coli culture and the infected E. coli culture in a 5 L fermentor (“fermentation parameters”) are kept constant as follows. Scaling up or down to allow for a smaller or larger scale fermentation follows these guidelines:
a. agitation of between 200 and 1,000 rpm, and in some embodiments between 300 and 600 rpm;
b. an energy source, such as, for example, glucose or glycerol, and optionally yeast extract, a buffering solution, trace elements, and thiamine. The media in the fermentor has a starting concentration of glucose or glycerol of between 3 and 7 grams per liter (L). When this energy source is almost depleted (about 3.5-7 hours after start of fermentation), additional glucose or glycerol is provided at a rate between 0.5-1.6 g/L/h, or alternatively 0.5-3.2 g/L/h (“the feed rate” The additional glucose or glycerol may be accompanied by Mg²⁺, yeast extract and a buffering solution;
c. dissolved oxygen (“DO”) of between 20% and 40% including, for example, 20, 25, 30, 35, or 40%, controlled by a cascaded control loop, wherein the primary response to a change in DO is to alter the agitation rate (between 200 and 1000 rpms), and the secondary response to a change in DO is to supplement the air flow line with pure oxygen. In another exemplary embodiment, the air flow rate discussed in step (d) is not kept constant, but is adjusted as needed depending on the DO;
d. an air flow rate of 0.5-2.0 volume/volume/minute (vvm);
e. a pH of not less than 6.5; and
f. temperature between 30° C. and 39° C. including, for example, 30, 31, 32, 33, 32, 35, 36, 37, 38, or 39° C.

The dissolved oxygen is kept constant between 20% and 40% by continually measuring the dissolved oxygen content, and adjusting the amount of agitation accordingly. An automated feedback loop can be used for monitoring DO and adjusting agitation. For example, if the dissolved oxygen threatens to fall below 20%, agitation may be increased. If the dissolved oxygen threatens to rise past 40%, agitation may be decreased. If agitation cannot maintain the dissolved oxygen content between 20 and 40%, pure oxygen may be added. Alternatively, the dissolved oxygen percentage may also be adjusted by placing the fermentation tank under pressure.

If the pH falls below 6.5, base is added.

Foaming may be controlled by bolus additions of antifoam, such as, for example, 20% Hydrite 3721 antifoam, at approximately 0 hrs, 4.5 hrs, 18 hrs, 24 hrs, 30 hrs, and 40 hrs, as needed. Antifoam may be added via syringe and needle through the septum port.

The filamentous bacteriophage (e.g., M13) are harvested between 20 and 28 hours after inoculation of filamentous bacteriophage into the fermentor or when the concentration of filamentous bacteriophage is at least 4×10¹² filamentous bacteriophage per milliliter (mL).

In some embodiments, methods for producing a culture of filamentous bacteriophage having a concentration of at least 4×10¹² filamentous bacteriophage per mL according to exemplary process 3 comprise the steps of: A method for producing a culture of filamentous bacteriophage having a concentration of at least 4×10¹² filamentous bacteriophage per mL comprising the steps of:
a) growing an E. coli culture in a first shake flask for 20 to 28 hours, wherein the E. coli express an F pilus,
b) transferring a volume of E. coli culture from the first flask into a second shake flask, wherein the volume to be transferred is between 0.5 and 20% of the volume of media to be transferred into;
c) growing the E. coli culture in the second shake flask for 20 to 28 hours;
d) transferring a volume of E. coli culture from the second flask into a fermentor, wherein the volume to be transferred is between 0.5 and 20% of the volume of media to be transferred into, and infecting the E. coli culture with between 2.0×10⁸ and 3.0×10⁸ filamentous bacteriophage per mL of starting medium;
e) maintaining the temperature of the fermentor between 30° C. and 39° C., the dissolved oxygen content between 20% and 40%, the pH above 6.5, the air flow between 0.5 and 2.0 vvm, and the agitation between 300 and 1200 rpms (e.g., 300-600 rpms, 600-1200 rpms, or 1000-1200 rpms; higher rpms may be appropriate for smaller fermentors, and vice versa);
f) adding glucose at a concentration of between 8 and 12 grams per liter at about 5.25 to 7.25 hours after diluting the E. coli culture into the fermentor at a rate between 2.5 and 5.5 grams per hour; and
g) harvesting the filamentous bacteriophage 20-28 hours after the start of step (e) when the bacteriophage have a titer of at least 4×10¹² bacteriophage per mL.

### Exemplary Process 4

A fourth process, in which bacteria are cultured in two stages before addition to the fermentor, comprises at least the following steps. This exemplary process can involve use of a relatively low amount of phage with respect to the amount of bacteria in the culture at the time of phage addition.

A bacterial E. coli strain, such as, for example, JM109, JM107 or other strains of E. coli expressing an F pilus, are grown in a shake flask in an incubated shaker at 37° C. and 250 rpm for 6 to 30 hours, including, for example, 6, 7, 8, 9, 10, 11, 12, 13, 14, 15, 16, 17, 18, 19, 20, 21, 22, 23, 24, 25, 26, 27, 28, 29, or 30 hours. The media may be any media known to support growth of E. coli, such as, for example, Minimal media, Luria Bertani (LB) and Terrific Broth (TB).

After 6 to 30 hours, typically 20 to 24 hours, a volume of E. coli culture from the first shake flask is transferred into a second shake flask. Typically, the volume of E. coli culture to be transferred is between 0.5 and 20% of the volume of media to be transferred into, assuming the OD₆₀₀ of the E. coli culture is between 0.5 and 10 units. For example, 2.5-100 mL of E. coli culture may be transferred into a second shake flask containing 500 mL of media (assuming an OD₆₀₀ between 0.5 and 10 units). The media in the first and second shake flask may be any media known to support growth of E. coli, such as, for example, Minimal media, Luria Bertani (LB) and Terrific Broth (TB).

In the event a fermentor or other means to generate a high cell density culture is used instead of the shake flask for the first pre-culture and assuming the OD₆₀₀ of the E. coli culture is between 0.5 and 200 units, the volume to be transferred may be between 0.01 and 20% of the volume of media to be transferred into.

The second shake flask is grown for about 6 to 30 hours, including, for example, 6, 7, 8, 9, 10, 11, 12, 13, 14, 15, 16, 17, 18, 19, 20, 21, 22, 23, 24, 25, 26, 27, 28, 29, or 30 hours.

After 6 to 30 hours, a volume of E. coli culture from the second shake flask is transferred into a fermentor. Typically, the volume of E. coli culture to be transferred is between 0.5 and 20% of the volume of media to be transferred into, assuming the OD₆₀₀ of the E. coli culture is between 0.5 and 10 units. For example, 2.5-100 mL of E. coli culture may be transferred into a fermentor containing 500 mL of media (assuming an OD₁₀₀ between 0.5 and 10 units). The fermentor comprises modified Riesenberg media (see Examples), or media with similar ingredients.

In the event a fermentor or other means to generate a high cell density culture is used instead of a shake flask for the first or second pre-culture and assuming the OD₆₀₀ of the E. coli culture is between 0.5 and 200 units, the volume to be transferred would be between 0.01 and 20% of the volume of media to be transferred.

The conditions or parameters for growth of the E. coli culture and the infected E. coli culture in a 5 L fermentor (“fermentation parameters”) are maintained as follows. Scaling up or down to allow for a smaller or larger scale fermentation follows these guidelines:
g. agitation of between 200 and 1,000 rpm, and in some embodiments between 300 and 600 rpm;
h. an energy source, such as, for example; glucose or glycerol, and optionally yeast extract, a buffering solution, trace elements, and thiamine. The media in the fermentor has a starting concentration of glucose or glycerol of between 3 and 7 grams per liter (L). When this energy source is almost depleted (about 3.5-7 hours after start of fermentation, for example, at a time ranging from 4 to 7, 4 to 6.5, 4 to 6, 4.5 to 7, 4.5 to 6.5, 4.5 to 6, 5 to 7, 5 to 6.5, or 5 to 6 hours after start of fermentation); additional glucose or glycerol is provided at a rate between 0.5-1.6 g/L/h, or alternatively 0.5-3.2 g/L/h (“the feed rate”). The additional glucose or glycerol may be accompanied by Mg² yeast extract and a buffering solution;
i. dissolved oxygen (“DO”) of between 20% and 40% including; for example, 20, 25, 30, 35, or 40%, controlled by a cascaded control loop, wherein the primary response to a change in DO is to alter the agitation rate (between 200 and 1000 rpms), and the secondary response to a change in DO is to supply oxygen at a higher concentration, e.g., by supplementing the air flow line with pure oxygen. In another exemplary embodiment, the air flow rate discussed in step (d) is not kept constant, but is adjusted as needed depending on the DO. In another exemplary embodiment, altering the tank pressure is used as a supplemental DO control strategy (e.g., when a stainless steel system is utilized);
j. an air flow rate of 0.5-2.0 volume/volume/minute (vvm);
k. a pH of not less than 6.5; and
l. temperature between 30° C. and 39° C. including, for example, 30, 31, 32, 33, 32, 35, 36, 37, 38, or 39° C.

The dissolved oxygen is maintained between 20% and 40% by continually measuring the dissolved oxygen content, and adjusting the amount of agitation accordingly. An automated feedback loop can be used for monitoring DO and adjusting agitation. For example, if the dissolved oxygen threatens to fall below 20%, agitation may be increased. If the dissolved oxygen threatens to rise past 40%, agitation may be decreased. If agitation cannot maintain the dissolved oxygen content between 20 and 40%, pure oxygen may be added. Oxygen may be supplemented into the 0.5-2.0 vvm air flow by the opening of a valve (controlled by the digital control unit as part of the cascade control loop). Alternatively, the dissolved oxygen percentage may also be adjusted by placing the fermentation tank under pressure.

If the pH falls below 6.5, base is added.

A glucose or glycerol feed is initiated at between 3.5 and 7 hours after transfer to the fermentor, including, for example, 3.5, 4.0, 4.5, 5.0, 5.5, 6.0, 6.5 or 7 hours. In some embodiments, the feed is initiated at a time ranging from 4 to 7 hours, 4 to 6 hours, from 4.5 to 6 hours, from 5 to 6 hours, from 5.5 to 6 hours, from 4 to 5.5 hours, from 4 to 5 hours, from 4.5 to 5.5 hours, or from 4.5 to 5 hours. Glucose or glycerol is provided between 0.5 and 1.6 g/L/h, including, for example, 0.5, 0.6, 0.7, 0.8, 0.9, 1.0, 1.1, 1.2, 1.3, 1.4, 1.5 and 1.6 g/L/h, or alternatively 0.5-3.2 g/L/h.

Once the E. coli culture in the fermentor reaches an OD₆₀₀ between 45 and 60, the E. coli culture is infected with filamentous bacteriophage, such as M13. The titer of the bacteriophage inoculums can be between 5×10⁴ and 2×10⁶ phage per milliliter (mL) of culture starting volume per unit OD₆₀₀, e.g., 1×10⁶ phage per milliliter (mL) of culture starting volume per unit OD₆₀₀ or 1×10⁵ phage per milliliter (mL) of culture starting volume per unit OD₆₀₀. In some embodiments, the filamentous bacteriophage used in the inoculation step are produced by growing them in a shake flask or other non-fermentor vessel. Prior to addition to the fermentor, the filamentous bacteriophage (e.g., M13) can be diluted in an appropriate buffer such as PBS, for example, giving 50 mL of phage in PBS which is then added to a fermentor culture (e.g., of volume 5 L). The agitation is reduced to 100 rpm while pumping the bacteriophage into the fermentor at between 8 and 12 mL per minute over a 3 to 7 minute period. The air flow is maintained at 0.5-2.0 vvm and feed is continued as per the “fermentation parameters” throughout.

A pipette, syringe, or serological pipette may be used to inoculate the E. coli culture. Alternatively, the filamentous bacteriophage may be pumped in, transferred by gravity, or transferred by other means, from a suitable container or bag through an addition port.

After the bacteriophage have been added, the agitation is continued for 1 to 3 minutes at about 100 rpm. Agitation is then stopped, leaving aeration and feed on, for about 20 to 40 minutes. Agitation is then resumed and ramped from about 200 to about 500 rpm over 10 to 40 minutes. After this step, DO control is resumed per the fermentation parameters.

The filamentous bacteriophage are harvested between 40 and 48 hours after start of the E. coli in the shake flask, or 20 to 24 hours after inoculation of filamentous bacteriophage into the fermenter or when the concentration of filamentous bacteriophage is at least 4×10¹² filamentous bacteriophage per milliliter (mL).

The yield of filamentous bacteriophage may be at least 1×10¹³ to 9×10¹³ phage per mL, or 1×10¹⁴ to 9×10′⁴ phage per mL.

Foaming may be controlled by bolus additions of antifoam, such as, for example, 20% Hydrite 3721 antifoam, at various times, such as approximately 0 hrs, 4.5 hrs, 18 hrs, 24 hrs, 30 hrs, and 40 hrs, as needed. Antifoam may be added via syringe and needle through the septum port or pumped in through an addition bottle or other suitable reservoir.

TABLE 2
Comparison of Four Exemplary Methods for Producing
High Titer Filamentous Bacteriophage such as M13

| Step # | Process 1 | Process 2 | Process 3 | Process 4 |
|---|---|---|---|---|
| 1 | Grow bacteria in | Grow bacteria in | Grow bacteria in | Grow bacteria in |
| | shake flasks | shake flask for | shake flask for | shake flask for |
| | until OD₆₀₀ = | 20-48 hours | 20-28 hours | 20-48 hours |
| | between 1 and 20 | | | |
| 2 | Transfer | Transfer a volume of | Transfer a volume of | Transfer a volume of |
| | bacteria to | bacterial culture from | bacterial culture from | bacterial culture from |
| | fermentor by | step 1 into a second | step 1 into a second | step 1 into a second |
| | diluting 1:40 into | shake flask. The | shake flask. The | shake flask. The |
| | a starting media | volume to transfer is | volume to transfer is | volume to transfer is |
| | | between 0.5 and | between 0.5 and | between 0.5 and |
| | | 20% of the volume of | 20% of the volume of | 20% of the volume of |
| | | media to be | media to be | media to be |
| | | transferred into, | transferred into, | transferred into, |
| | | assuming the OD₆₀₀ | assuming the OD₆₀₀ | assuming the OD₆₀₀ |
| | | of the bacterial | of the bacterial | of the bacterial |
| | | culture is between | culture is between | culture is between |
| | | 0.5 and 10 units. | 0.5 and 10 units. | 0.5 and 10 units. |
| 3 | N/A | Grow the second | Grow the second | Grow the second |
| | | flask for 20-28 | flask for 20-28 | flask for 20-28 |
| | | hours | hours | hours |
| 4 | N/A | Transfer a volume of | Transfer a volume of | Transfer a volume of |
| | | bacterial culture from | bacterial culture from | bacterial culture from |
| | | step 3 into a | step 3 into a | step 3 into a |
| | | fermentor. The | fermentor. The | fermentor. The |
| | | volume to transfer is | volume to transfer is | volume to transfer is |
| | | between 0.5 and | between 0.5 and | between 0.5 and |
| | | 20% of the volume of | 20% of the volume of | 20% of the volume of |
| | | media to be | media to be | media to be |
| | | transferred into, | transferred into, | transferred into, |
| | | assuming the OD₆₀₀ | assuming the OD₆₀₀ | assuming the OD₆₀₀ |
| | | of the bacterial | of the bacterial | of the bacterial |
| | | culture is between | culture is between | culture is between |
| | | 0.5 and 10 units. | 0.5 and 10 units. | 0.5 and 10 units. |
| | | | AND infect with M13 | |
| | | | 50 ul of phage bank | |
| | | | at a concentration of | |
| | | | 2.8 × 10¹⁴ phage | |
| | | | particles/mL (1.4 × | |
| | | | 10¹³ phage total or | |
| | | | 3.5 × 10¹² phage/L) | |
| 5 | T = 30-39° C., Dissolved Oxygen (“DO”) between 20 and 40% (cascaded with | | | |
| | agitation [200-1000 rpm] and optional pure oxygen), pH not below 6.5, air flow | | | |
| | rate of 0.5-2.0 vvm | | | |
| 6 | Glucose at a | Glucose at a starting concentration of 3-7 g/L; additional | | |
| | starting | glucose added at about 4-7 hours after start of fermentation at | | |
| | concentration of | a rate of 0.5-1.6 grams per liter per hour, or alternatively | | |
| | 8-12 g/L; | 0.5-3.2 g/L/h. | | |
| | additional | | | |
| | glucose added | | | |
| | at about 4-7 | | | |
| | hours after start | | | |
| | of fermentation | | | |
| | at a rate of | | | |
| | 0.5-1.6 grams per | | | |
| | liter per hour, or | | | |
| | alternatively | | | |
| | 0.5-3.2 g/L/h. | | | |
| 7 | Infection with | Infection with | Infection at step 4 | Infection with M13 |
| | M13 at OD₆₀₀ | M13 at OD₆₀₀ | (time of inoculation, | at OD₆₀₀ between |
| | between 50 and | 50 +/− 5, at which | no specific process | 45 and 60, at |
| | 70, at which | point the agitation | parameters for | which point the |
| | point the | is reduced to 100 | infection, standard | agitation is |
| | glucose feed is | rpm whilst | fermentation | reduced to 100 |
| | stopped. Once | pumping in phage | parameters) | rpm whilst |
| | a DO spike | (8-12 mL/min | | pumping in phage |
| | greater than | added over a 3-7 | | (8-12 mL/min |
| | 40% is noted, | min period). Air | | added over a 3-7 |
| | the agitation is | flow maintained | | min period). Air |
| | stopped. Air | at 0.5-2.0 vvm, | | flow maintained at |
| | flow is | and feed is on. | | 0.5-2.0 vvm, and |
| | maintained at | After phage are | | feed is on. |
| | 0.5-2.0 vvm. | added, agitation | | After phage are |
| | M13 is added | is continued for | | added, agitation is |
| | neat or diluted | 1-3 mins at about | | continued for 1-3 |
| | in PBS. | 100 rpm. | | mins at about 100 |
| | A 30 minute | Agitation is then | | rpm. Agitation is |
| | incubation with | stopped, and | | then stopped, and |
| | no agitation is | aeration and feed | | aeration and feed |
| | followed by | remain on for | | remain on for |
| | restarting | 20-40 minutes. After | | 20-40 minutes. After |
| | agitation with | 20-40 minutes, | | 20-40 minutes, |
| | normal cascade | agitation is | | agitation is ramped |
| | control. Once | ramped from | | from 200-500 rpm |
| | the DO | 200-500 rpm over | | over 10-40 minutes |
| | concentration | 10-40 minutes then | | then back to |
| | exceeds 20%, | back to normal | | normal cascade |
| | the feed is started. | cascade control. | | control. |
| | 2.5 × 10⁸ M13 per | 2.5 × 10⁸ M13 per | | 1 × 10⁶ M13 per mL |
| | mL culture | mL culture | | culture starting |
| | starting volume | starting volume | | volume per unit |
| | per unit OD₆₀₀ | per unit OD₆₀₀ | | OD₆₀₀ |

| | | | |
|---|---|---|---|
| 8 | Harvest time = 40-48 hours after | Harvest time = | Harvest time = |
| | inoculation of bacteria in the | 20-28 hours after | 40-48 hours after |
| | fermentor or 20-24 hours after | inoculation of M13 | inoculation of |
| | inoculation of M13 into the fermentor | into the fermentor or | bacteria in the |
| | or when the M13 concentration is | when the M13 | fermentor or 20-24 |
| | greater than 4 × 10¹² M13 per mL. | concentration is | hours after |

| | |
|---|---|
| greater than 4 × 10¹² | inoculation of M13 |
| M13 per mL. | into the fermentor |
| | or when the M13 |
| | concentration is |
| | greater than 4 × |
| | 10¹² M13 per mL |
| | or 1 × 10¹³ M13 per |
| | mL. |

Each of the four processes may be conducted on a small or large scale. 1 liter to 100,000 liters are encompassed. Volumes and concentrations may be scaled from the numbers described above.

It is to be understood that both the foregoing and following description are exemplary and explanatory only and are not restrictive of the invention, as claimed.

### Example 1

### Large Scale Production of High Titer Wild Type M13 Phage

One exemplary process for producing high concentrations of filamentous bacteriophage consists of the following protocol: E. coli are grown in a shake flask until the culture reaches an OD₆₀₀ of between 1 and 4 (usually between 20-24 h). The E. coli culture is transferred to a fermentor, the feed is initiated, and the culture is allowed to grow. Once the E. coli reach an OD₆₀₀ of 55+/−5, the culture is infected with filamentous bacteriophage from a virus stock suspension. Growth continues for another 20-24 h and the E. coli cells are removed by centrifugation.

In this experiment, E. coli JM109 were obtained from a frozen glycerol stock culture and grown in M9 culture in baffled Erlenmeyer Flasks.

Glycerol stocks of the E. coli host strain were generated per the following E. coli glycerol stock preparation protocol:
1) Thaw a 1 mL glycerol stock tube containing E. coli strain of choice at 37° C. and vortex briefly to mix.
2) Use 1 mL to inoculate 50 mL of M9 minimal medium (see ingredients below) in a 250 mL flask (2% inoculum). Other media, such as, for example, Luria Bertani (LB) and Terrific Broth (TB) may also be used.
3) Incubate the culture at 37° C. with shaking (250 rpm) for 16 hours (or overnight (“o/n”).
4) The following day or 16 hours later, 2.5 mL is used to inoculate each of two 1 liter flasks containing 250 mL of M9 minimal medium.
5) The flasks are incubated at 37° C. with shaking (250 rpm) to an OD₆₀₀ of 0.6-0.8. Samples are taken from a duplicate flask to measure the OD₅₀₀ to ensure the other flask is not contaminated during sampling. Discard the flask used to take measurements as soon as it reaches OD.
6) Once the duplicate flask has reached the target OD, take the other flask into the sterile hood and using a 50 mL transfer pipette, transfer the culture into a 500 mL centrifuge bottle (pre-sterilized, conical bottom). Use another bottle and insert to balance the centrifuge if necessary.
7) Harvest the E. coli cells by spinning the bottles and inserts at 4000 rpm for 20 minutes in a centrifuge such as Sorvall RC-3.
8) Return the centrifuge tube to the hood and use a large transfer pipette (e.g. 50 mL) attached to the electronic pipettor to carefully remove the supernatant.
9) Add 250 mL of fresh M9 minimal medium supplemented with 15% (w/v) glycerol (cell culture grade) to the pellet.
10) Use a large transfer pipette (e.g. 50 mL) to gently resuspend the cells.
11) As soon as most of the cells are resuspended, use a small transfer pipette (10 mL) to completely resuspend the cells and ensure there are no cell clumps visible.
12) Using a 5 or 10 mL transfer pipette, aliquot 200, 1 mL samples into sterile glycerol stock (2 mL Nunc Cryovials) tubes, ensuring the resuspended culture is mixed regularly so the cells do not have time to settle. Aliquot a small number (20-30) and then snap-freeze so the cells do not settle in the tubes.
13) Snap-freeze the small batches of tubes by placing in a suitable container with dry ice pellets. Try to ensure the tubes stay upright during freezing.
14) Once the samples have been frozen, place in a labeled box and store at −80° C.

TABLE 3
Recipe for M9 minimal medium

| | | |
|---|---|---|
| 10x M9 salts | 100 | mL/L |
| 1M MgSO₄ | 2 | mL/L |
| 1M CaCl₂ | 100 | μl/L |
| 20% Glucose | 20 | mL/L |
| 200x Vitamins | 5 | mL/L |
| 1M Thiamin | 500 | μl/L |
| 100 mM FeSO₄ | 1 | mL/L |
| 1000x Trace Minerals | 1 | mL/L |

Preparation of Stock Solutions: 10× salts, MgSO₄, CaCl₂ and glucose were made and autoclaved separately. Similarly, vitamins, thiamin and trace minerals were made and filter sterilized separately. FeSO₄ is prepared and used as fresh as possible and filter sterilized prior to use. To make M9 medium, first add 10× salts and water and autoclave. Cool, then add all other ingredients.

TABLE 4
Vitamins, Trace Minerals, and M9 Salts
200x Vitamins

| | | |
|---|---|---|
| Thiamin | 1 | g/L |
| Biotin | 200 | mg/L |
| Choline Cl | 200 | mg/mL |
| Folic acid | 200 | mg/L |
| Niacinamide | 200 | mg/L |
| Pantothenate | 200 | mg/L |
| Pyridoxal | 200 | mg/L |
| Riboflavin | 20 | mg/L |

Trace Minerals

| | | |
|---|---|---|
| CuSO₄•5H₂O | 0.2497 | g/100 mL |
| MnSO₄•H₂O | 0.1690 | g/100 mL |
| ZnSO₄•7H₂O | 0.2875 | g/100 mL |

10x M9 salts

| | | |
|---|---|---|
| Na₂HPO₄•7H₂O | 128 | g/L |
| KH₂PO₄ | 30 | g/L |
| NaCl | 5 | g/L |
| NH₄Cl | 10 | g/L |

The volume of E. coli added to the shake flask is typically 2% of the final working volume of the fermentor. Thus, for a 5 L production, a 500 mL baffled Erlenmeyer flask containing 100 mL of sterile M9 medium is inoculated aseptically with 1.0 mL of stock E. coli suspension from a thawed 1 mL culture cryovial between 0.6 and 0.8 OD₆₀₀ units. Typically, at least two flasks are set up in parallel and monitored for growth and purity prior to inoculation into the fermentor.

The shake flask is incubated at 37° C. and agitated at 250 rpm in an incubated shaker with a stroke length of 1″ (e.g., New Brunswick Scientific Innova 44). The shake flasks are incubated for 16-24 h until the OD₆₀₀ is between 1-4. Flasks are checked microscopically for contamination before inoculating the production fermentor. One of the flasks was selected as the inoculum based on suitable OD₆₀₀ and absence of contamination.

Fermentor Preparation—Materials: New Brunswick Scientific Bioflo3000 bioreactor or equivalent equipped with a 7.5 L (5 L working volume) vessel; New Brunswick Scientific Biocommand operating software or equivalent and historian; 4 L defined growth medium, such as, for example, modified Riesenberg media as described herein, supplemented with yeast extract at 50 g/L; 1 L nutrient feed bottle; Base reservoir with NH₄OH; Antifoam reservoir with A204 defoamer or similar; Silicone tubing.

The fermentor was set up using the following control parameters:

TABLE 5
Fermentor control parameters

| Loop | Sensor | Actuator | Range | | Feedback |
|---|---|---|---|---|---|
| Agitation | tachometer | Motor driven | 200-1000 | rpm | DO control |
| | | impeller with 2 | | | loop cascade, |
| | | Rushton | | | 1^{st} response |
| | | turbines | | | |
| Dissolved | Mettler Toledo | Agitation and | 40% ± 5% | | DO control |
| Oxygen (“DO”) | Polarographic | oxygen | | | |
| | DO probe | supplementation | | | |
| | | (if needed) | | | |
| pH* | Mettler Toledo | Base peristaltic | 6.5-0.02 | | pH |
| | Gel pH probe | pump, 28% | | | |
| | | NH₄OH | | | |
| Temperature | RTD | Cold water and | 37 ± 0.l° | C. | Culture |
| | | internal heat | | | temperature |
| | | lamp | | | |
| Aeration | Mass flow | Manual gas flow | 5 ± 0.5 | l/min | DO control |
| | controller | needle valve, | | | loop cascade, |
| | | automated | | | 2^{nd} response |
| | | solenoid valve | | | |
| | | for PID | | | |
| | | controlled O₂ | | | |
| | | supplementation | | | |
| Nutrient feed | | Single speed | 5.5-22 | mL/h | Manual or |
| | | peristaltic pump - | | | supervised |
| | | rate controlled | | | control on |
| | | by duty cycle | | | timed step |
| | | | | | profile. |

| | | | | |
|---|---|---|---|---|
| Antifoam | Level probe | Single speed | NA | Manual or |
| | | peristaltic pump | | supervised |
| | | | | control |

*pH is controlled with base (ammonium hydroxide) only there is no control when the setpoint goes above pH 6.5 (e.g. with a feedback loop to an acid pump)

The online parameters were controlled and logged by a bioreactor controller. Supervisory software may also be used.

4 L of modified Riesenberg medium (see, Riesenberg et al., Journal of Biotechnology, 20 (1991) 17-28) and the modifications in Table 6) was added to the fermentor.

TABLE 6
Modified Riesenberg media and Feed Solution

| | Concentration | | Concentration | |
|---|---|---|---|---|
| | in Standard | | in modified | |
| Component | Riesenberg Medium^{a} | | RiesenbergMedium | |
| KH₂PO₄ | 13.3 | g/L | 13.3 | g/L |
| (NH₄)₂HPO₄ | 4.0 | g/L | n/a | |
| (NH₄)₂SO₄ | n/a | | 4.0 | g/L |
| Citric Acid | 1.7 | g/L | 1.7 | g/L |
| MgSO₄•7H₂O | 1.2 | g/L | 0.5 | g/L |
| Riesenberg Trace | 10 | mL/L | n/a | |
| metal solution | | | | |
| Trace metal solution A | n/a | | 1 | mL/L |
| Trace metal solution B | n/a | | 10 | mL/L |
| Thiamine HCl | 4.5 | mg/L | 47 | mg/L |
| Glucose•H₂O | 27.5 | g/L | 5 | g/L |
| Antifoam (Ucolab N115) | 0.1 | mL/L | n/a | |
| Antifoam (Hydrite 3721) | n/a | | 0.06 | mL/L |
| Feed solution | | | | |
| MgSO₄•7H₂O | 19.7 | g/L | 5 | g/L |
| Glucose•H₂O | 770 | g/L | 500 | g/L |

| NH₃ | 25% | n/a | |
|---|---|---|---|
| Yeast extract | n/a | 50 | g/L |
| K₂HPO₄ | n/a | 10 | g/L |
| KH₂PO₄ | n/a | 2.1 | g/L |

^{a}Riesenberg et al., Journal of Biotechnology, 20 (1991) 17-28.

TABLE 7
Trace Metal Solutions

| | | |
|---|---|---|
| | Concentration | Concentration |
| | in Standard | in modified |
| Component | Riesenberg Medium^{a} | Riesenberg Medium |

Riesenberg Trace
Metal Solution

| | | | |
|---|---|---|---|
| Fe(III) citrate | 6 | g/L | n/a |
| MnCl₂•4H₂O | 1.5 | g/L | n/a |
| Zn(CH₃COO)₂2H₂O | 0.8 | g/L | n/a |
| H₃BO₃ | 0.3 | g/L | n/a |
| Na₂MoO₄•2H₂O | 0.25 | g/L | n/a |
| CoCl₂•6H₂O | 0.25 | g/L | n/a |
| CuCl₂•2H₂O | 0.15 | g/L | n/a |
| EDTA | 0.84 | g/L | n/a |

| Trace Metal | | | |
|---|---|---|---|
| Solution A | | | |
| Citric Acid | n/a | 3 | g/L |
| CoCl₂•6H₂O | n/a | 2 | g/L |
| MnCl₂•4H₂O | n/a | 12 | g/L |
| CuCl₂•2H₂O | n/a | 1.13 | g/L |
| H₃BO₃ | n/a | 2.5 | g/L |
| Na₂MoO₄•2H₂O | n/a | 2 | g/L |
| Trace Metal | | | |
| Solution B | | | |
| Fe(III) citrate | n/a | 6 | g/L |
| EDTA | n/a | 0.84 | g/L |
| Zn(CH₃COO)₂•2H₂O | n/a | 0.8 | g/L |

^{a}Riesenberg et al., Journal of Biotechnology, 20 (1991) 17-28.

A pH probe was calibrated and a 7.5 L fermentor was autoclaved at 121° C. and 15 psi for 40 minutes.

The following addition solutions are also prepared and sterilized in an autoclave at 122° C. for 30 minutes.

TABLE 8
Glucose addition solution

| | Manu- | | Product | Amount |
|---|---|---|---|---|
| Chemical | facturer | Grade | number | required |
| Glucose | USB/Pfanstiehl | TECH. GRADE | 14535 | 40 g |
| MgSO₄•7H₂O | Baker | ACS | 2500-05 | 4.8 g |

The following thiamine and base solution is prepared and filter sterilized using, for example, a 0.22 μm filter. Thiamine and base solutions can be stored for several months at −20° C., for example.

TABLE 9
Thiamine solution

| | Manu- | | Product | Amount |
|---|---|---|---|---|
| Chemical | facturer | Grade | number | required |
| Thiamine•HCl | Sigma | REAGENT | T4625-250 | 0.34 g |
| | | GRADE | | |
| DI water | Deionized | PURE | NA | QS to 10 mL |

TABLE 10
Base solution

| | Manu- | | Product | Amount |
|---|---|---|---|---|
| Chemical | facturer | Grade | number | required |
| Aqueous | Baker | ACS | 972133 | 500 mL |
| NH₄OH | | | | |

Trace elements are prepared as follows: Protocol for Making Trace Element Solution A (TES A):
1) Dissolve 3 g Citric Acid in 50 mL warm water.
2) Dissolve 2 g CoCl₂.6H₂O in 50 mL warm water. Add to solution 1.
3) Dissolve 12 g MnCl₂.4H₂O in 50 mL warm water. Add to solution 2.
4) Dissolve 1.13 g CuCl₂.H₂O in 50 mL warm water. Add to solution 3. Make up to 500 mL and boil.
5) Dissolve 2.5 g H₃BO₃ in 60 mL warm water.
6) Dissolve 1 g Citric Acid in 40 mL warm water. Add to solution 5. Boil and add to boiled solution 4.
7) Dissolve 2 g Na₂MoO₄.2H₂O in 50 mL warm water.
8) Dissolve 1 g Citric Acid in 50 mL warm water. Add to solution 7. Boil and add to solution 6.
9) Make up to 1 liter with water.
10) Filter sterilize and store at 4° C.

Protocol for Making Trace Element Solution B (TES B):
1) Dissolve 6 g Fe(III)citrate in 100 mL warm water.
2) Dissolve 0.84 g ethylene-dinitrilo-tetraacetic acid in 100 mL warm water.
3) Dissolve 0.8 g Zn(CH₃COO)₂.2H₂O in 100 mL warm water. Add to solution 2.
4) Add solution 3 to solution 1.
5) Make up to 1 liter with water.
6) Filter sterilize and store at 4° C.

An empty reservoir bottle is autoclaved at 122° C. for 30 minutes. The bottle may be equipped with a filter capped vent line and a dip tube connected to silicone tubing, the other end of which has a connector allowing quick aseptic connection to the fermentor base addition line. When cool, ammonium hydroxide was aseptically transferred into the reservoir.

TABLE 11
Feed solution

| | Manu- | | Product | Amount | |
|---|---|---|---|---|---|
| Chemical | facturer | Grade | number | required | |
| Glucose | USB/ | TECH. GRADE | 14535 | 500 | mL |
| | Pfanstiehl | | | | |
| MgSO₄•7H₂O | Baker | ACS | 2500-05 | 5 | g |

| Yeast extract | Bacto | TECH. GRADE | 288610 | 50 |
|---|---|---|---|---|
| KH₂PO₄ | Baker | ACS | 3246-05 | 10.0 |
| K₂HPO₄ | Fisher | ENZ. GRADE | BP363-1 | 2.1 |
| DI water | Deionized | PURE | NA | QS to 1 l |

TABLE 12
Optional Antifoam solution

| | | | | Exemplary |
|---|---|---|---|---|
| | Manu- | | Product | Amount |
| Chemical | facturer | Grade | number | required |
| Antifoam 204 | Sigma | Not specified | A6426 | 20 mL |
| Ethanol | Decon | 200 Proof | 2716 | 80 mL |

### Example 2

### Reactor/Fermentor Preparation

After initial cooling, the reactor was hooked up to the base unit and all probes and ancillary equipment, including feed, base and antifoam reservoirs were attached. Power, temperature control and air sparge were turned on and a probe to measure the dissolved oxygen was allowed to polarize for at least 2 hours, but normally overnight. Any type of air sparge may be used to maximize air dispersion and break up any bubbles.

The supervisory software was set up to log all control loops. In addition to the measured loops, two calculated loops: base totalizer and nutrient feed totalizer programs were set up to determine the amount of base and feed added by calculation of pump duty cycle.

When the medium was cool, prior to inoculation with filamentous bacteriophage, the following additions were added from the stock solutions prepared as described above:

TABLE 13
Additions to Media

| Addition | Amount |
|---|---|
| Glucose/MgSO4 solution | 250 mL |
| Thiamine solution | 0.5 mL |

The dissolved oxygen probe was calibrated immediately prior to inoculation. A medium blank sample was removed and retained in a sterile tube. A further sample was tested for pH with an off-line pH meter to check the rector pH probe calibration. Corrective action would be taken if the pH value is more than 0.1 units outside correct calibration.

### Example 3

### Fed Batch Fermentation

The following approximate control set-points are used during fermentation. If a parameter is threatening to increase or decrease from a set-point, corrective action, such as, for example, raising or lowering the temperature, adding base to raise the pH once it dips below 6.5, or increasing or decreasing agitation to increase or decrease the dissolved oxygen content is taken.

TABLE 14
Exemplary Set Points for Fed-Batch Fermentation

| Parameter | Set point | |
|---|---|---|
| Temperature | 37° | C. |
| pH | 6.5 | |
| Dissolved oxygen | 40% | |
| Gas flow rate | 5 | LPM |
| Agitation Rate | 200-1000 | rpm |

| | |
|---|---|
| % Oxygen | Set by dissolved oxygen |
| | (DO) cascade |

The fermentor was inoculated with the entire contents of one shake flask (OD₆₀₀ between 1-4) that was prepared and tested as outlined above. Transfer was done aseptically. A zero time point sample was removed. For this time point and for other samples taken during the fermentation the following tests were done:

TABLE 15
Time point tests during fermentation

| Parameter tested | Method | Additional Notes |
|---|---|---|
| Biomass concentration | Absorbance at 600 nm | Done for each sample |
| Glucose concentration | Diabetes test kit | Done for each sample |
| | (immediate)/HPLC | |
| | (retrospective) | |
| Metabolic products | HPLC | Optional, but desirable |
| Virus Count | ELISA | Done on samples taken |
| | | after virus infection |
| Protein visualization | SDS-PAGE (Coomassie) | Done on samples taken |
| | | after virus infection |

Growth and on-line data were regularly monitored. If the glucose is consumed, oxygen demand will drop rapidly as evidenced by a decrease in agitation rate and increase in DO concentration. This is the trigger to start the nutrient feed, and glucose or glycerol feeding is initiated. The initial nutrient feed rate is 5.5 mL/h and the following step change feed profile was used:

TABLE 16
Step Change Feed Profile

| | Feed Rate |
|---|---|
| Time | (mL/h, 5 L working volume) |
| Inoculation to consumption of batch | 0 |
| glucose (approx 6-9 h) | |
| Feed start + 0 h | 5.5 |
| Feed start + 6 h* | 11.0 |
| Feed start + 20 h | 16.5 |
| Feed start + 25 h | 22.0 |

*Except when feed is paused at virus addition, based on a 50% or 500 g/L glucose feed

The feed rates are not very high and thus oxygen demand is not excessive. Oxygen supplementation is optional, and often not required. Growth proceeds in a linear fashion as feed is added.

### Example 4

### Addition of M13

M13 filamentous bacteriophage are added to the culture when the culture OD₆₀₀ (OD) is 55±5. At the feed rates described above, this OD was attained between 20-24 hours after inoculation. M13 (prepared per the protocol provided below in “Virus glycerol stock preparation protocol”) was previously stored as a frozen suspension at −80° C. at a concentration of 2.8×10¹⁴ phage/mL.

The E. coli culture is infected with M13 at a rate of 2.5×10⁸ M13 per mL culture starting volume per unit OD. Thus, for a 5 L final culture volume, with a starting volume of 4 L and an infection OD of 50, 5×10¹³ M13 particles are used to infect the culture. For a 5 L fermentor, 178 μl of M13 stock solution at a stock concentration of 2.8×10¹⁴ phage/mL are required to infect the culture.

To calculate the amount of M13 stock solution to add, use the following equation: M13 to add (total phage)=2.5×10⁸ phage/OD600/mL multiplied by OD600 multiplied by volume (mL) or M13 to add (mL)=[2.5×10⁸ phage/OD600/mL multiplied by OD600 multiplied by volume (mL)] divided by phage concentration 2.8×10¹⁴/mL

The nutrient feed pump is stopped temporarily, and as the dissolved oxygen spikes (greater than 40%), the agitation is stopped. The air flow is kept constant at 1-1.25 vvm (corresponding to 4-5 L/min given the 4 L culture volume) and the virus suspension is aseptically added to the fermentor. The reactor is allowed to stand without agitation for 30 minutes before restarting agitation. Once agitation had been restarted and the dissolved oxygen concentration is above 20%, the feed pump is restarted at a rate as shown in Table 16.

### Example 5

### M13 Glycerol Stock Preparation Protocol

Stocks of M13 at 2×10¹⁴ phage/mL, in PBS supplemented with 15% (w/v) glycerol are prepared as follows: at 47 hours post inoculation of the fermentor, a 5 liter fermentor produces approximately 1×10¹³ phage/mL. With a final supernatant volume of 4 L there are ˜4×10¹⁶ phage particles produced. Before glycerol addition, the phage are concentrated to 2.82×10¹⁴ phage/mL. Assuming downstream recovery of 30%, the phage re concentrated to 50 mL.

Protocol for making M13 (or any type of filamentous bacteriophage) glycerol stocks:
1) Take a 1 mL glycerol stock of E. coli. Thaw at 37° C. and then vortex briefly to mix.
2) Use 500 μl to inoculate 50 mL of M9 minimal medium in a 250 mL flask.
3) Incubate at 37° C. with shaking (250 rpm) for 5-6 hours.
4) Use 10 mL of the culture to inoculate 250 mL of fresh M9 minimal medium in a 1 liter flask.
5) Incubate at 37° C. with shaking (250 rpm) for 16 hours (or o/n).
6) After the 16 hour incubation, use all 250 mL to inoculate the 5 liter fermentor, which contains 3.5 liters of Riesenberg medium (see below) supplemented with 1% (w/v) yeast extract.
7) Incubate the cells at 37° C., pH 6.5, DO 30% with feeding to an OD₆₀₀ of 55±5.
8) Stop the agitation of the fermentor and add M13 from a stock to 8.76×10¹¹ phage particles per OD₆₃₀ unit.
9) Incubate the M13 with the cells for 30 minutes without agitation and then continue the fermentor with agitation as usual.
10) 24 hours after infection, harvest the cells using a disk stack centrifuge (e.g., Whisperfuge) at maximum speed (˜12,000×g) and collect the supernatant.
11) Concentrate the supernatant to ˜200 mL with the 500 kDa hollow fiber and then diafilter with 10 volumes of PBS. Concentrate down to a final volume of ˜50 mL.
12) Filter-sterilize the sample through a 0.2 μm filter (e.g., NALGENE) and store at 4° C. Do an ELISA to determine concentration.
13) Once the concentration is known, dilute with PBS to 2.82×10¹⁴ phage/mL.
14) Add 15% (w/v) glycerol (cell culture grade) and mix thoroughly.
15) Filter-sterilize the sample through a 0.2 μm filter.
16) Using a 5 or 10 mL transfer pipette, aliquot 200, 1 mL samples into sterile glycerol stock (2 mL Nunc Cryovials) tubes, ensuring the remainder of the sample is mixed regularly so the phage does not have time to settle. Aliquot a small number (20-30) and then snap-freeze.
17) Snap-freeze the small batches of tubes by placing in a suitable container with dry ice pellets. Try to ensure the tubes stay upright during freezing.
18) Once the samples have been frozen, place in a labeled box and store at −80° C.

The temperature of the starting material before filtration and the temperature of the concentrated material after filtration is monitored to ensure that the temperature has not risen too much during processing. Room temperature is also monitored.

When the culture has been infected for 24 h the fermentation is terminated. The nutrient feed is stopped, at which point a DO spike is observed. The reactor is cooled to 5-10° C.

### Example 6

### Harvesting M13

M13 are harvested by first removing the host E. coli cells by centrifugation. Floor centrifuges, a disk stack centrifuge (e.g., Whisperfuge) and a Sharples continuous centrifuge have all been used successfully. Alternatively, Tangential Flow Filtration (TFF) is used. Centrifugation may be done at approximately 12,000×g or equivalent in a continuous centrifuge.

After centrifugation, storage at 4° C. is acceptable. The bacteriophage are stable for at least two weeks at 4° C., but storage can lead to increased microbial load, and so holding at this stage should be minimized.

### Example 7

### Experimental Results from Exemplary Process

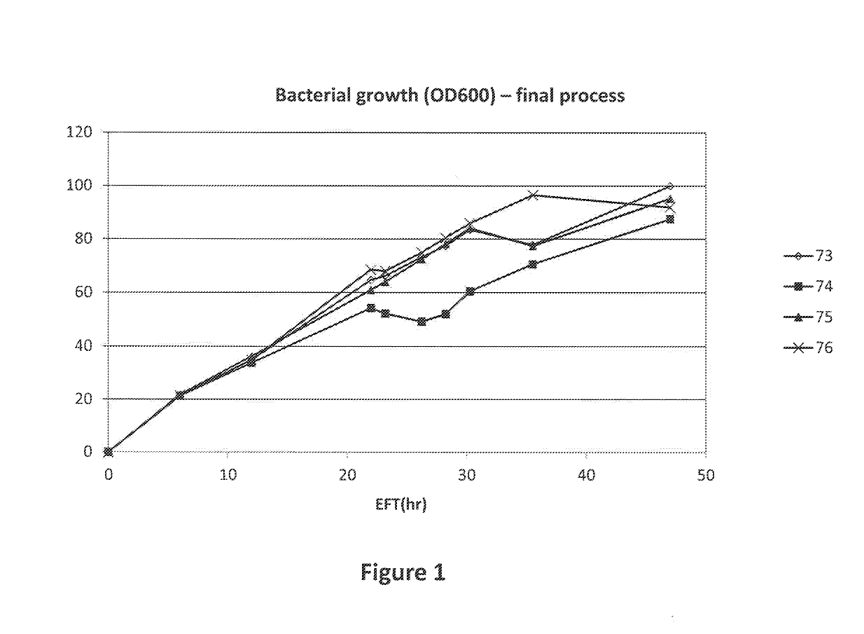

Process 1 was run on a 5 L scale in four replicates (FIG. 1; raw fermentation data below). Defined medium with yeast extract and 10 g/L glucose was used in the batch phase, along with a feed containing 50% glucose, yeast extract and salts. A cell-free phage suspension was added at an OD₆₀₀ of 55±5 at a level of 2.5×10⁸ phage/mL culture starting volume multiplied by OD₆₀₀. Cultures were grown for at least 24 h after infection with continual feeding. Growth reproducibility was achieved (FIG. 1).

In this experiment, the actual infection ODs were 64.7, 54.2, 61 and 68.6. The reactors were all infected at 22 hours after the initial E. coli culture was transferred to the fermentor.

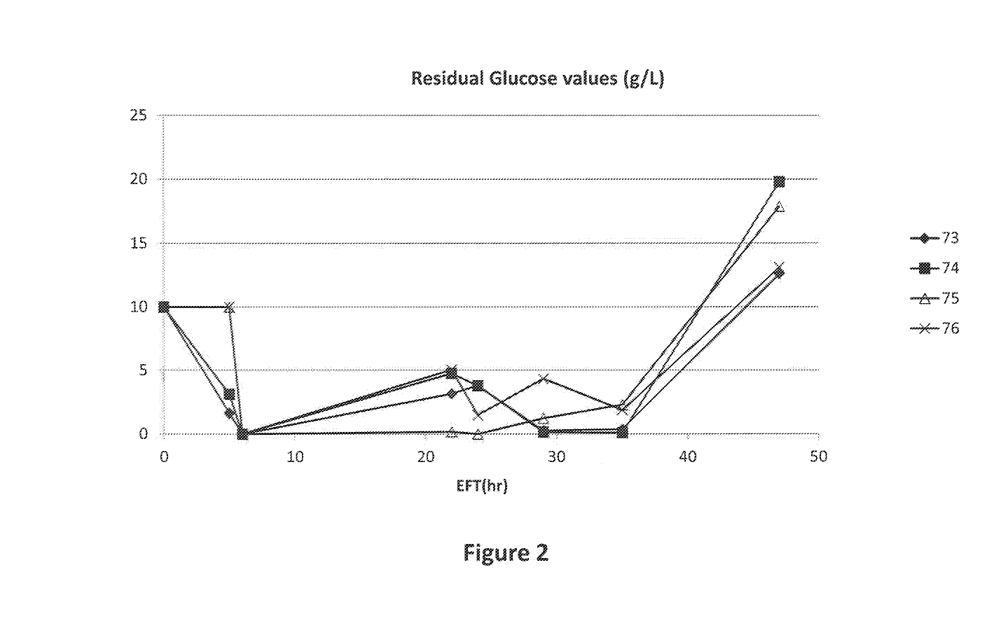

Substrate (glucose) concentration was monitored (FIG. 2). The substrate was initially consumed during the batch phase and was well controlled for the first 24 h of feeding. However, late in the feeding stage, possibly due to stress as more virus was produced and the E. coli cellular machinery was taxed, glucose consumption was reduced and substrate accumulated in the medium. This occurred despite the volumetric feed rate remaining constant. Thus, the dilution rate constantly decreased.

An ELISA was done to measure the phage produced over time. The results show a correlation between virus concentration and culture growth. In one specific culture, the final phage yield was 1.4×10¹³ phage/mL, but the average across the four cultures was higher at 6.9×10¹³ phage/mL.

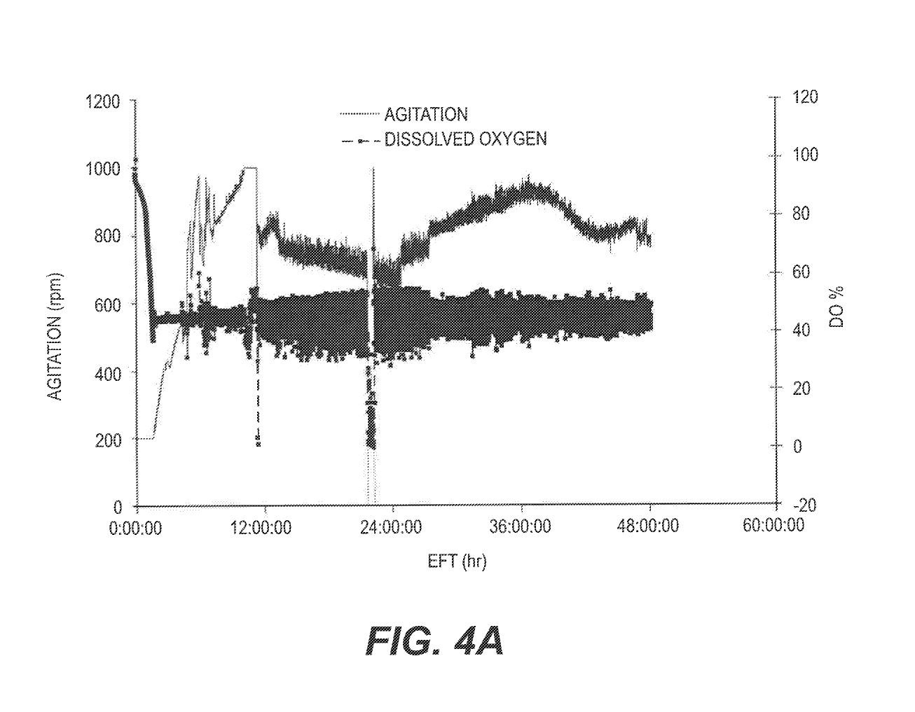

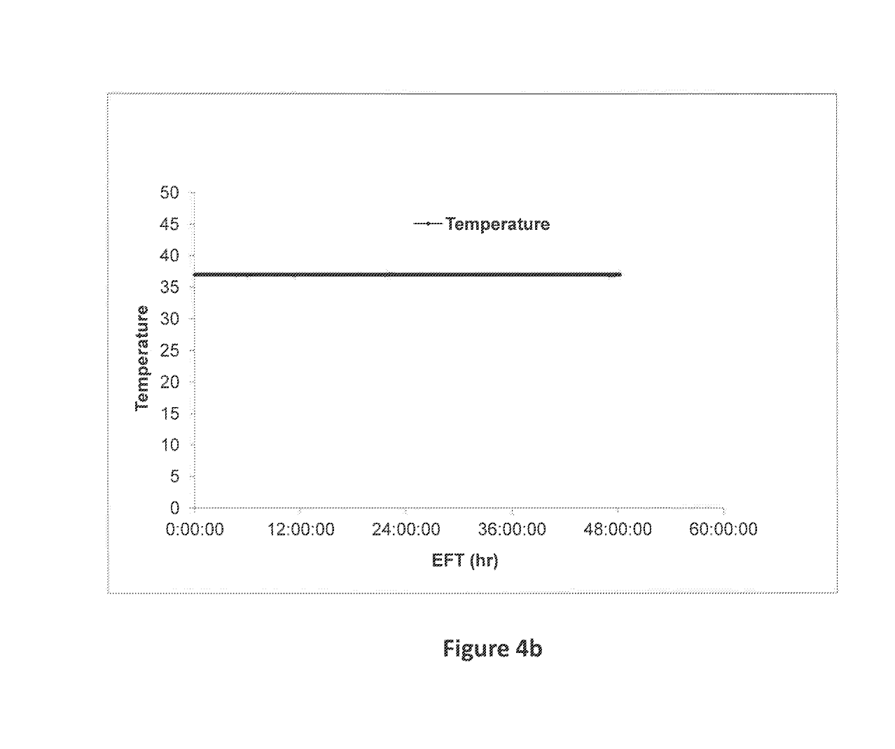

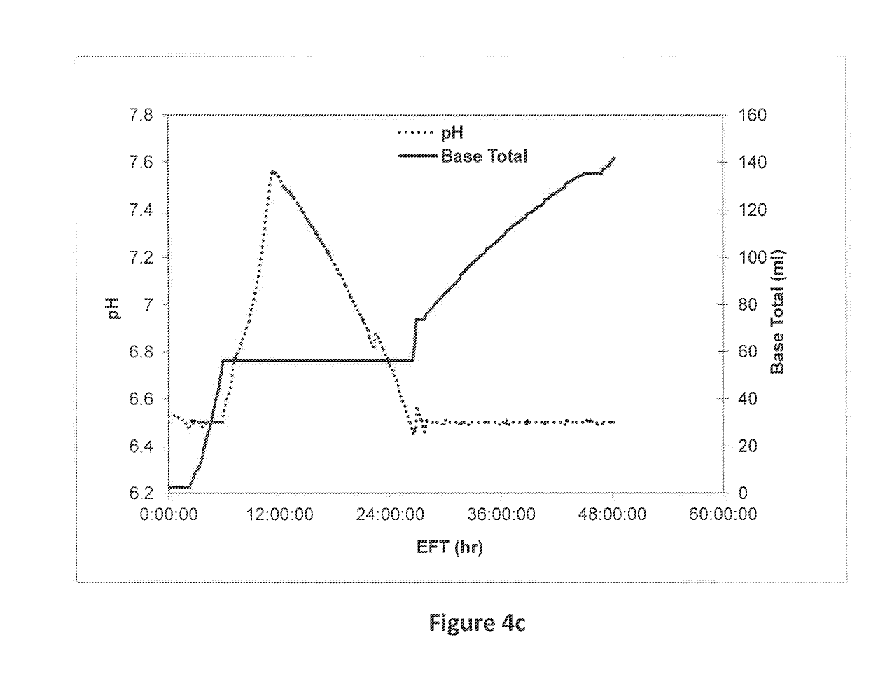

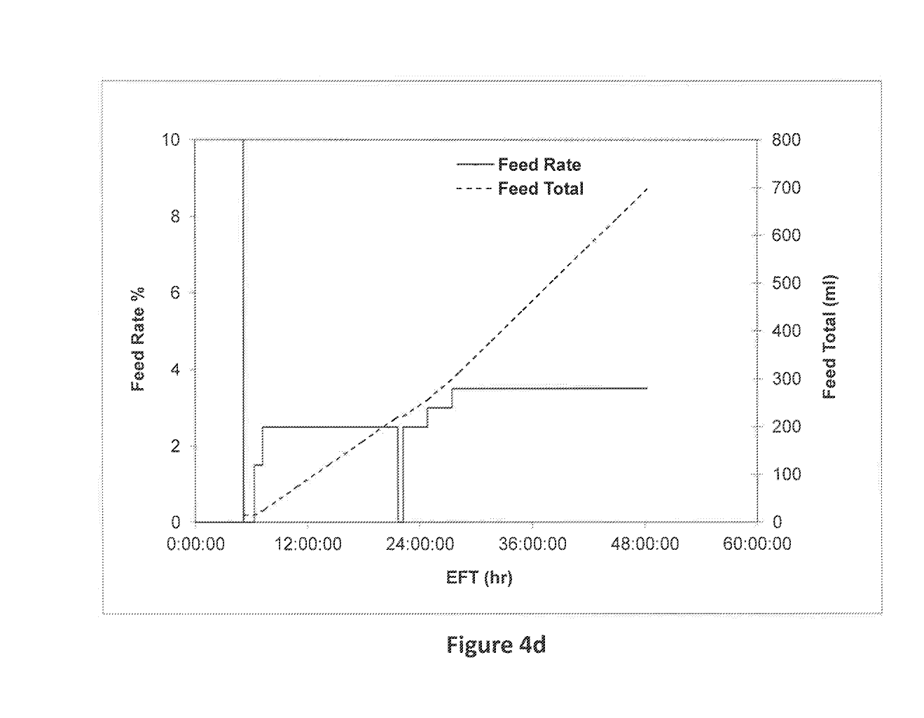

On-line process data for the fed-batch fermentations is shown in FIG. 4.

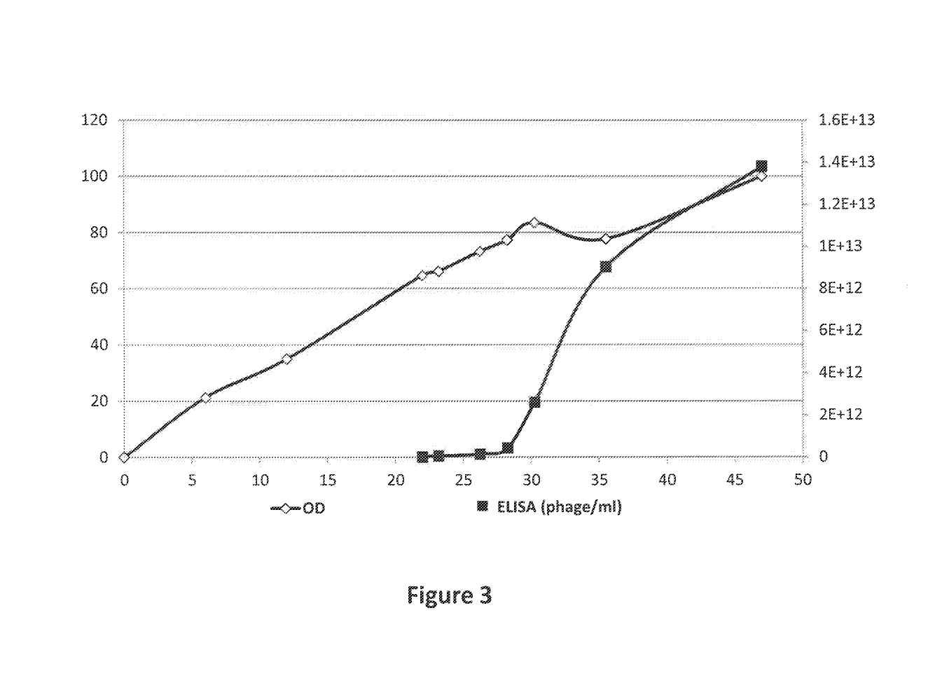

TABLE 17
Raw data for FIGS. 1, 2 and 3
Optical Density

OD values

| EFT(hr) | 73 | 74 | 75 | 76 |
|---|---|---|---|---|
| 0 | 0 | 0 | 0 | 0 |
| 6 | 21.3 | 21.3 | 21.8 | 21.6 |
| 12 | 35 | 33.8 | 36.3 | 34.9 |
| 22 | 64.7 | 54.2 | 61 | 68.6 |
| 23.2 | 66.2 | 52.2 | 64 | 68 |
| 26.25 | 73.3 | 49.2 | 72.5 | 75 |
| 28.25 | 77.4 | 52 | 78.2 | 80.5 |
| 30.25 | 83.6 | 60.5 | 84.1 | 86 |
| 35.5 | 77.8 | 70.6 | 77.4 | 96.6 |
| 47 | 100 | 87.6 | 95.3 | 92 |

Residual Glucose

| | Glucose Concentration | | | |
|---|---|---|---|---|
| EFT(hr) | 73 | 74 | 75 | 76 |
| 0 | 10 | 10 | 10 | 10 |
| 5 | 1.64 | 3.12 | 10 | 10 |
| 6 | 0 | 0 | 0 | 0 |
| 22 | 3.15 | 4.75 | 0.18 | 5.05 |
| 24 | 3.79 | 3.78 | 0 | 1.49 |
| 29 | 0.26 | 0.15 | 1.23 | 4.33 |
| 35 | 0.37 | 0.1 | 2.29 | 1.87 |
| 47 | 12.6 | 19.8 | 17.9 | 13.1 |

Phage counts

| time | OD | ELISA (pfu/mL) |
|---|---|---|
| 0 | 0 | |
| 6 | 21.3 | |
| 12 | 35 | |
| 22 | 64.7 | 1.82E+10 |
| 23.2 | 66.2 | 6.31E+10 |
| 26.25 | 73.3 | 1.48E+11 |
| 28.25 | 77.4 | 4.37E+11 |
| 30.25 | 83.6 | 2.62E+12 |
| 35.5 | 77.8 | 9.04E+12 |
| 47 | 100 | 1.38E+13 |

### Example 8

### ELISA for Detection and Quantitation of Wild Type M13 Phage

The following relates to the specific detection and quantification of intact M13 wild type phage using trap ELISA (enzyme-linked immunosorbent assay).

Intact M13 phage express both p3 (5 copies at the tip of the phage to promote attachment of the phage to bacterial F-pilus) and p8 (2,800 copies which serve as the major coat protein) proteins. Employing an antibody trap and detection assay that requires both proteins ensures that the assay measures whole, assembled phage. The M13 particles are detected and quantified by sandwich ELISA using two different antibodies. The M13 particles are captured (“trapped”) by anti-p3 monoclonal antibody and detected by anti-p8 monoclonal antibody conjugated to horseradish peroxidase (HRP).

TABLE 18
Materials

| Material | Type | Source |
|---|---|---|
| Capture Antibody: | Anti-M13 Phage | Exalpha |
| | Coat Protein (p3). | Biologicals, Inc. |
| | Mouse Monoclonal | Catalog number: |
| | Antibody E1. | Z115M. |
| Secondary Antibody | HRP/Anti-M13 (p8) | GE Healthcare. |
| | Monoclonal | Catalog number. |
| | Conjugate. | 27-9421-01. |
| Blocking Agent | Albumin, Bovine | Sigma. Catalogue |
| | Serum, Fraction V, | number A3059. |
| | Approx. 99%. | |
| Wash Buffer | Phosphate Buffered | Gibco. Catalogue |
| | Saline pH 7.4 10X. | number 70011. |
| | Polyoxyethylene- | Sigma. Catalog |
| | sorbitan | number P-1379. |
| | monolaurate | |
| | (Tween 20). | |
| Substrate | o- | Sigma. Catalog |
| | Phenylenediamine | number P5412- |
| | (OPD). | 50TAB. |
| ELISA Plates | F16 Maxisorp | Nalge Nunc |
| | Loose. Nunc- | International. |
| | Immuno Module. | Catalog number |
| | | 469914. |
| Plate Reader | VERSAmax | Molecular |
| | microplate reader | Devices. |
| | with SOFTmax Pro | |
| | software (v 4.3.1). | |
| Purified Phage | CsCl-purified | N/A |
| for Standard | production lot | |
| Curve | phage. Stored at 1 × | |
| | 10¹⁴ phage/mL. | |
| Plate Washer | Mulitdrop DW. | Thermo |
| | | Labsystems. |

100 μl of Anti-M13 p3 monoclonal antibody diluted 1:500 (2 μg/mL final concentration) in PBS was added to a 96 well ELISA plate and incubated for 2.5 hours at 37° C.

The plates were washed with 350 μl per well of Wash Buffer (PBS/0.05% Tween 20) 5 times. The plates were tapped on a paper towel after every wash.

The plates were blocked by adding 350 μl per well of 5% (w/v) BSA in PBS and incubated overnight at room temperature or 37° C. If the plates were not going to be used immediately the following day, they were stored at 4° C. with the BSA present. If the plates were going to be used immediately, the BSA was washed out and the empty plates were stored at either 4° C. or −20° C.

The plates were next washed 5 times in 350 μl per well of Wash Buffer (PBS/0.05% Tween 20) 5 times. The plates were tapped on a paper towel after every wash.

A standard curve was prepared by diluting the M13 stock solution (usually 1×10¹⁴ phage/mL) to 2×10¹⁰ phage/mL in PBS. 100 μl was added per well in duplicate. 2×10¹⁰ phage/mL was diluted two-fold in PBS (to 1×10¹° phage/mL) and 100 μl added per well in duplicate. The two-fold dilution was repeated six times, each time adding 100 μl per ell of stock in duplicate. 100 μl of PBS was added to four wells as a blank. The plate was incubated at 37° C. for 2 hours. The range of the standard curve is 2×10¹⁰-1.6×10⁸ phage/mL.

To prepare the unknown samples, the samples were diluted in the range of 2×10¹⁰-1.6×10⁸ phage/mL (to fall within the standard curve). 3-5 serial dilutions in PBS were necessary. 100 μl of each dilution was added to the plate in duplicate, and then incubated for 2 hours at 37° C.

The plates were washed with 350 μl per well of Wash Buffer (PBS/0.05% Tween 20) 5 times. The plates were tapped on a paper towel after every wash.

The bacteriophage were detected with the anti-M13 phage tail protein p8, HRP conjugate antibody. 100 μl of anti-M13 phage tail protein p8 monoclonal antibody, HRP conjugate diluted 1:5000 in 3% (w/v) BSA/PBS was added per well and then incubated at 37 CC for 1 hour.

The plates were then washed with 350 μl per well of Wash Buffer (PBS/0.05 Tween 20) 5 times. The plates were tapped on a paper towel after every wash.

The plates were developed by adding 100 μl of substrate per well (20 mg OPD in 10 mL of 50 mM citrate buffer pH 5.0 and 4 μl of H₂O₂). The reaction was stopped after 5 minutes with 50 μl of 4 M HCl. The A490 of each well was measured using SOFTmax PRO software. A four parameter-fit was used to plot the standard curve.

TABLE 19
Results of a typical standard curve.

| Standard Number | Concentration (phage/mL) |
|---|---|
| 1 | 2.0 × 10¹⁰ |
| 2 | 1.0 × 10¹⁰ |
| 3 | 5.0 × 10⁹ |
| 4 | 2.5 × 10⁹ |
| 5 | 1.3 × 10⁹ |
| 6 | 6.3 × 10⁸ |
| 7 | 3.1 × 10⁸ |
| 8 | 1.6 × 10⁸ |

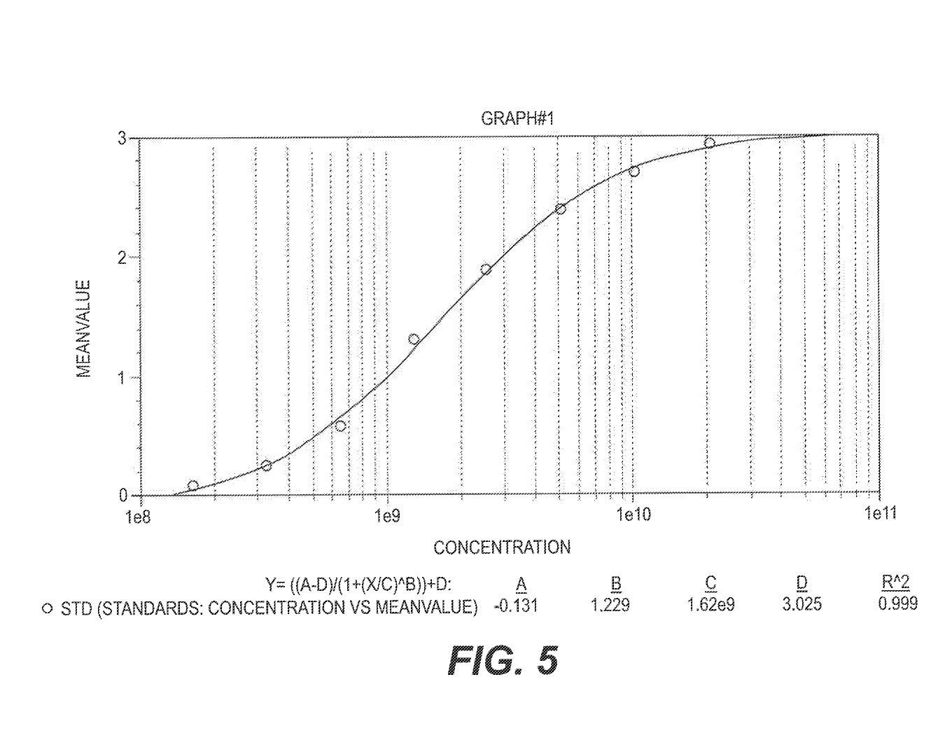

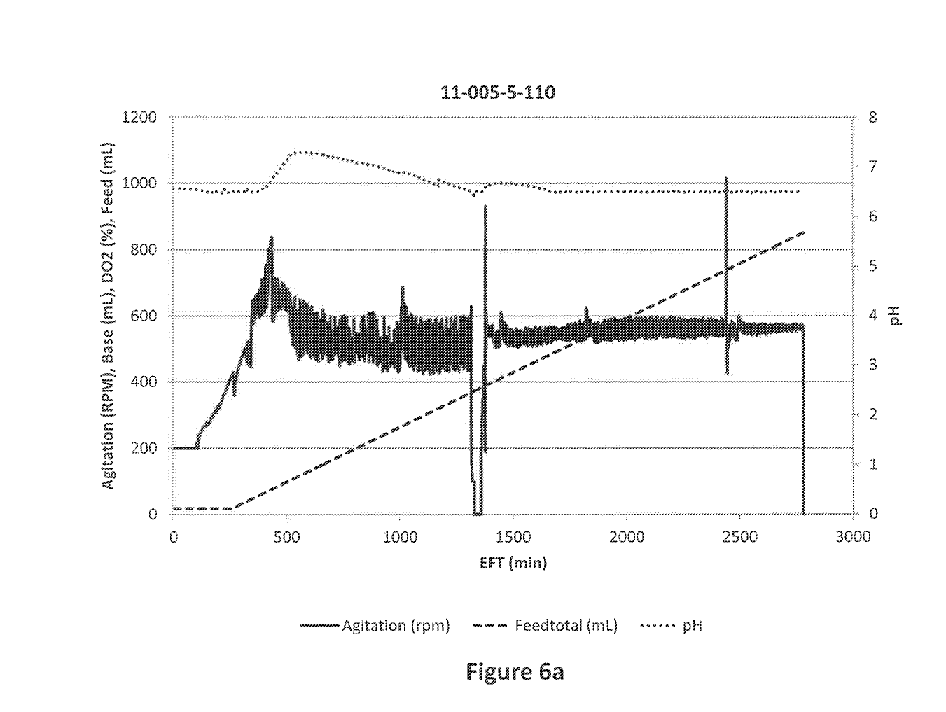

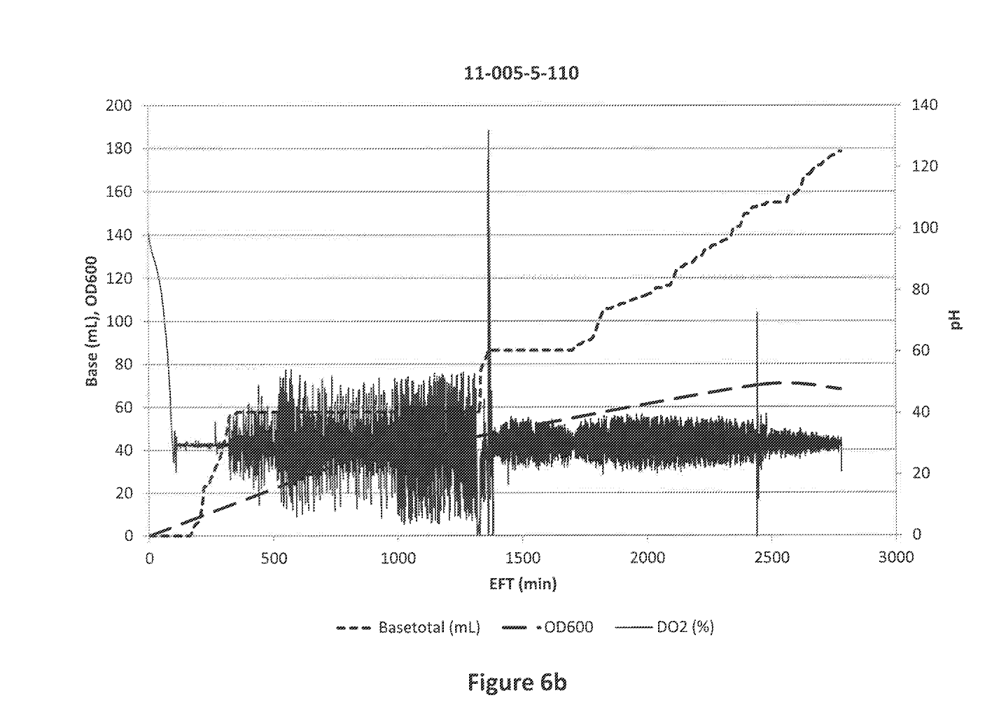

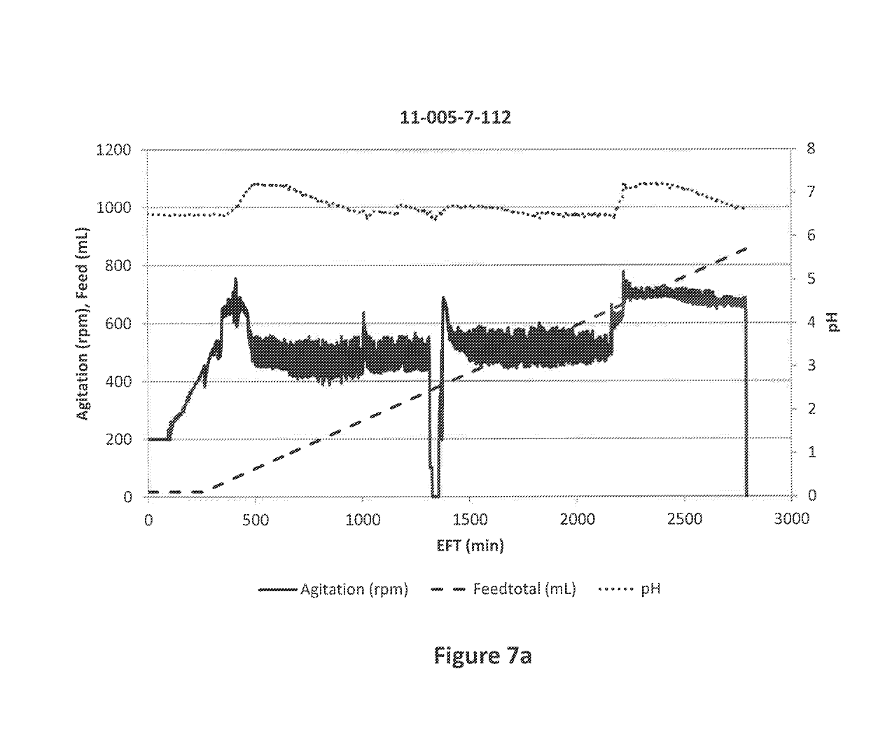

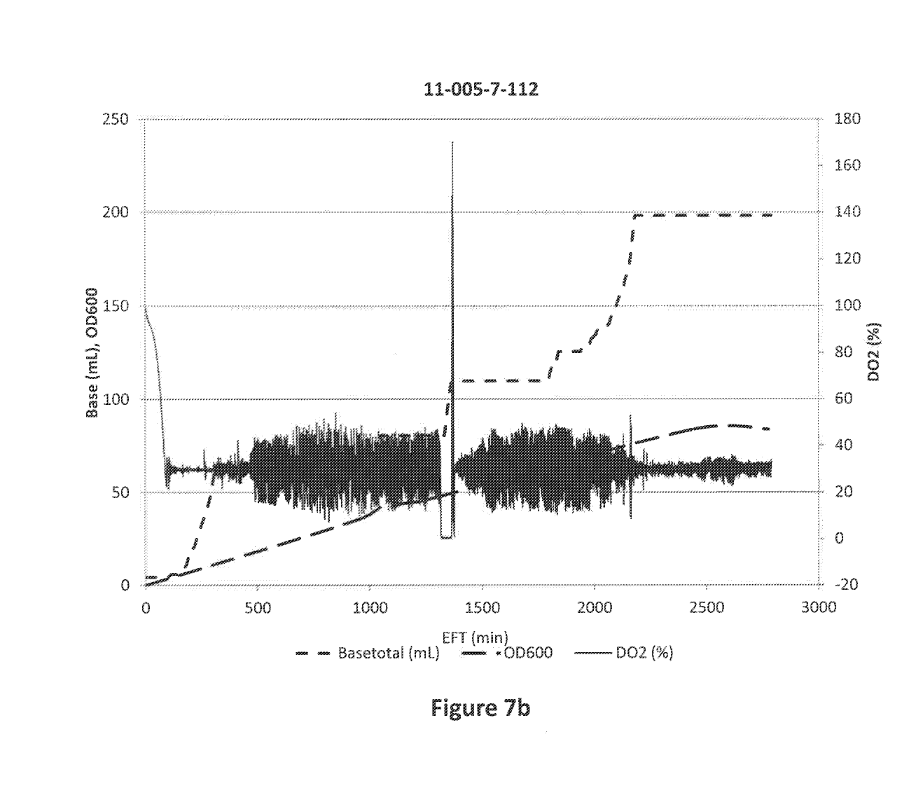

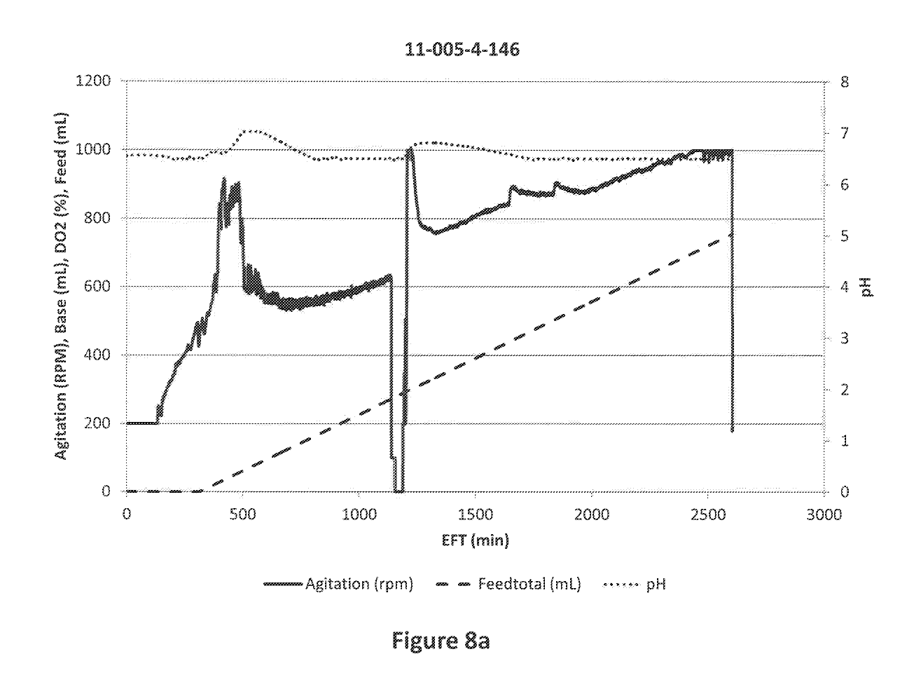

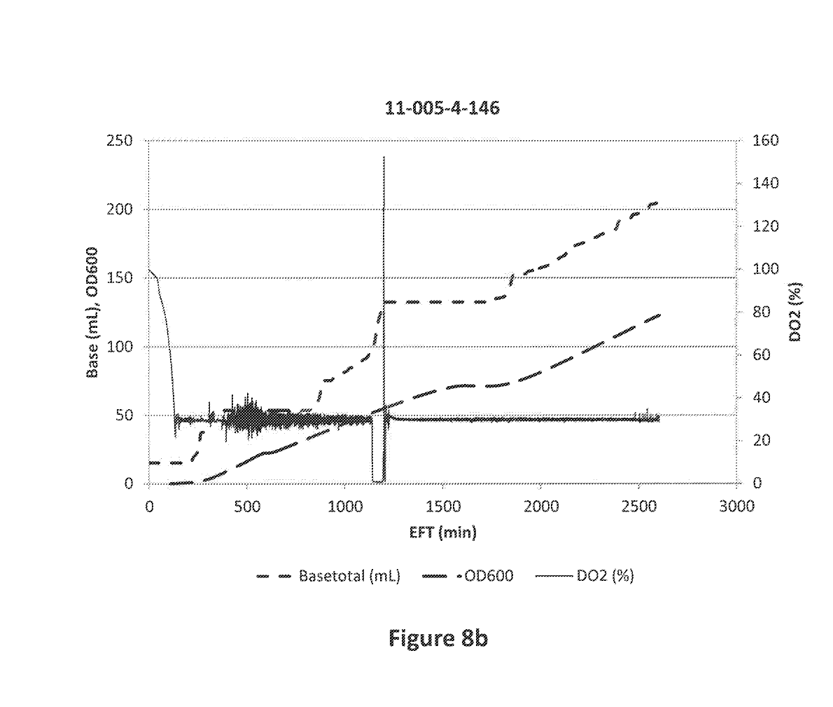

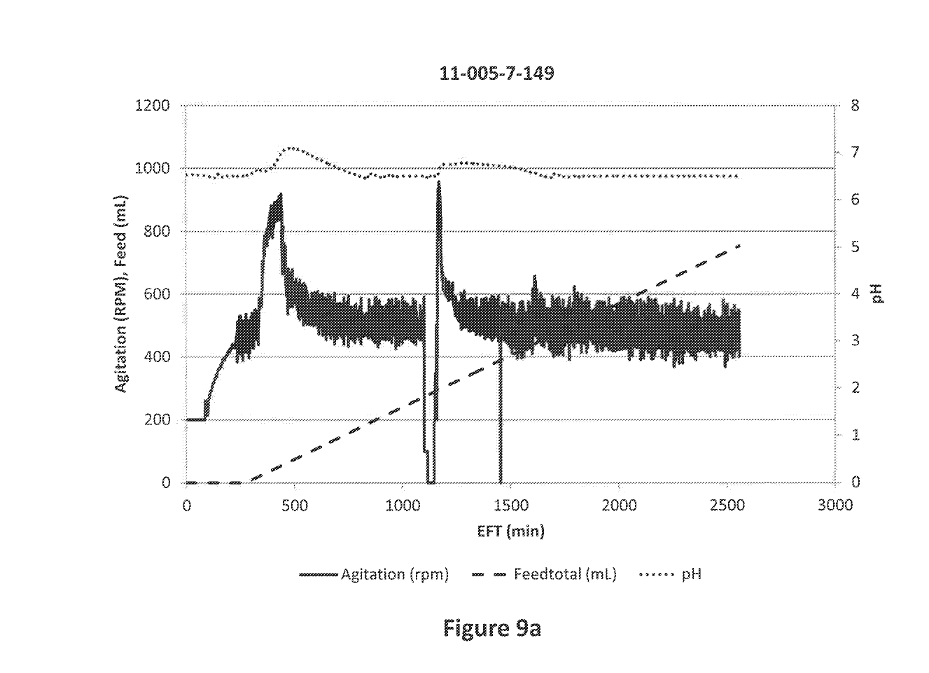

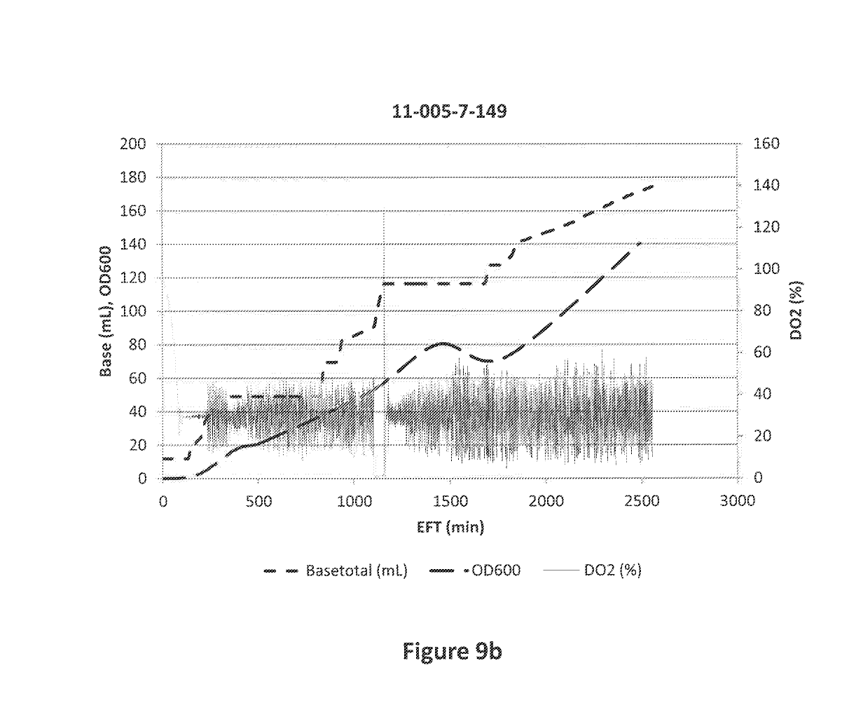

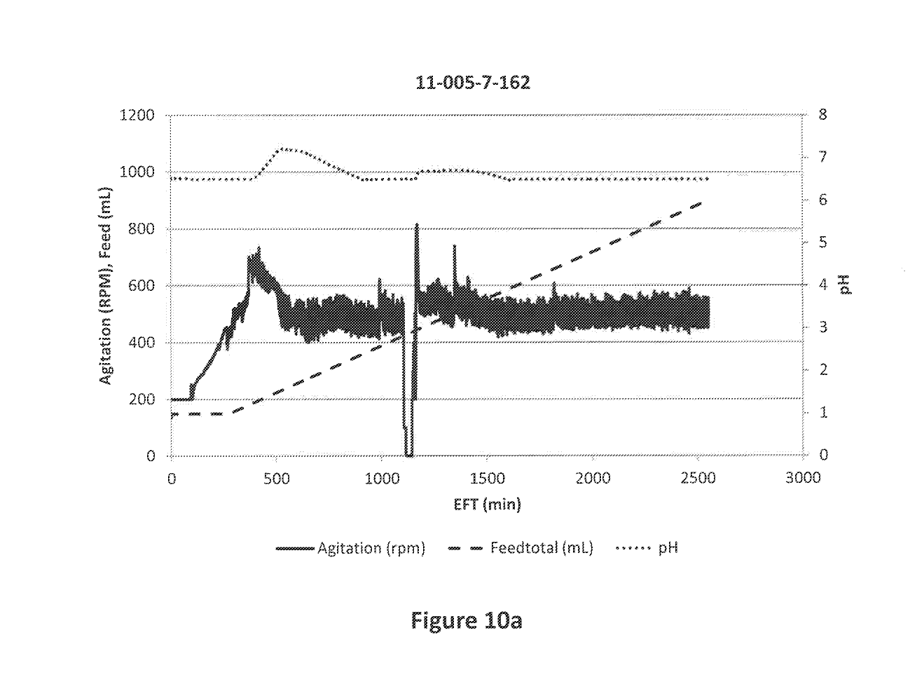

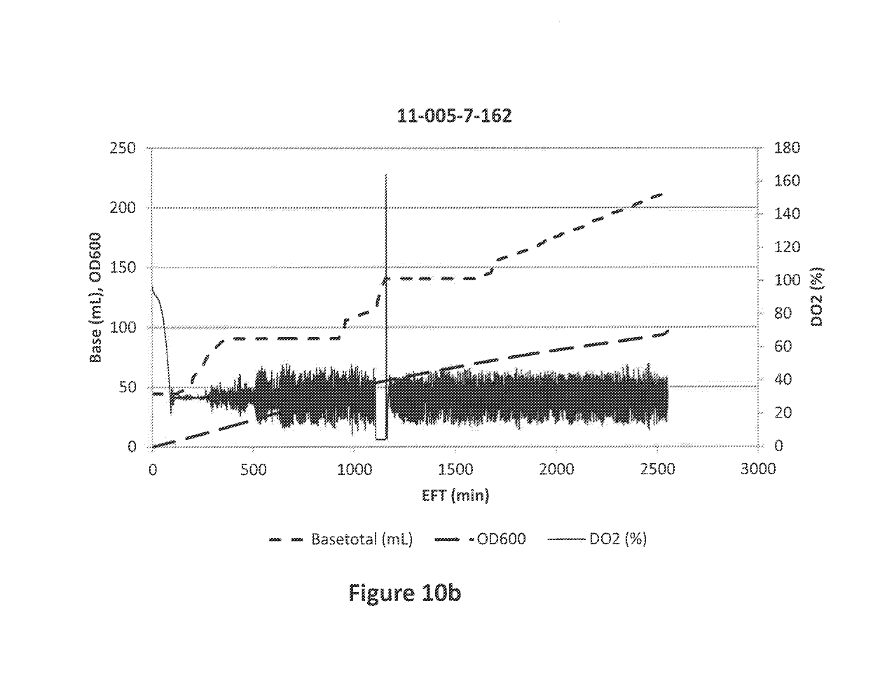

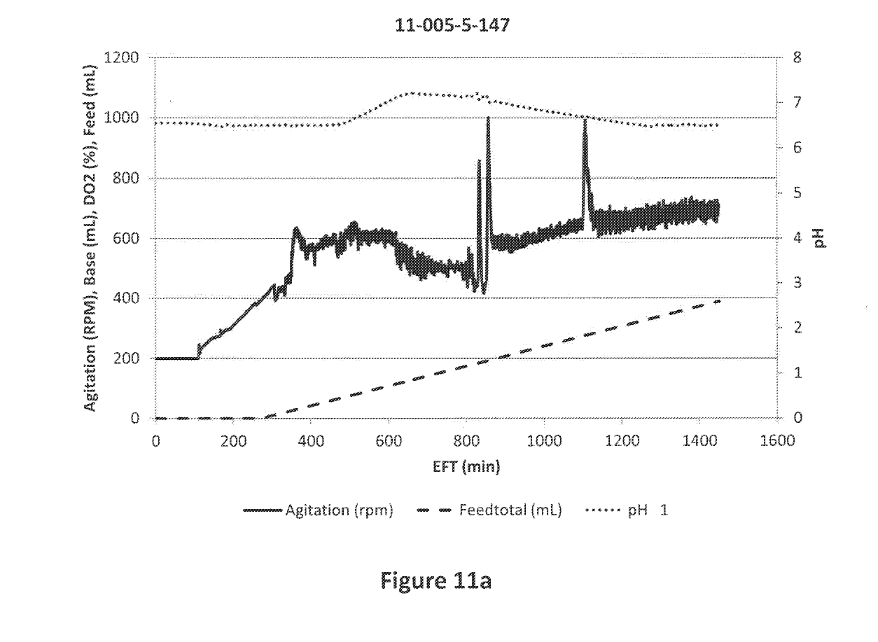

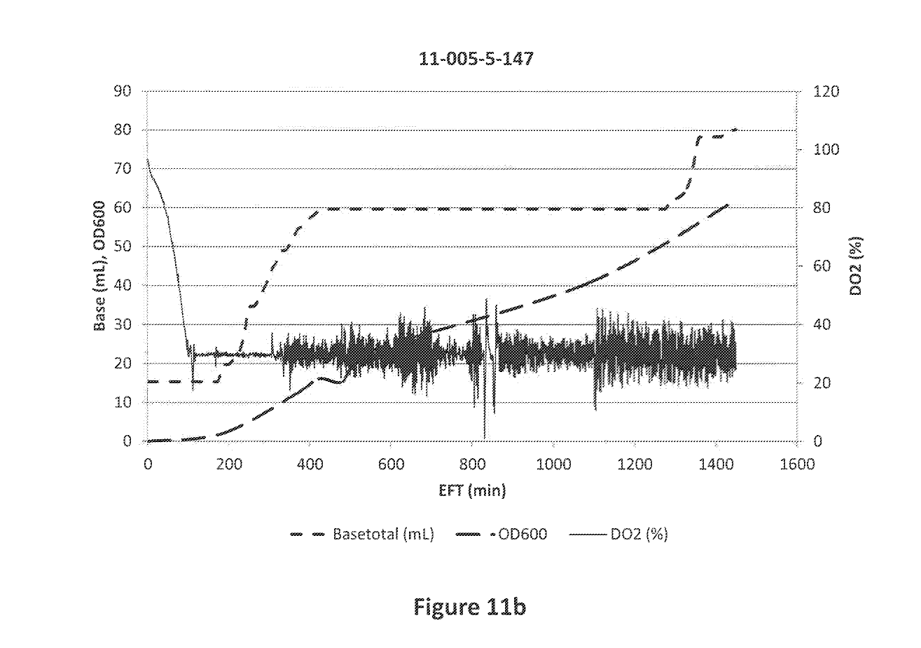

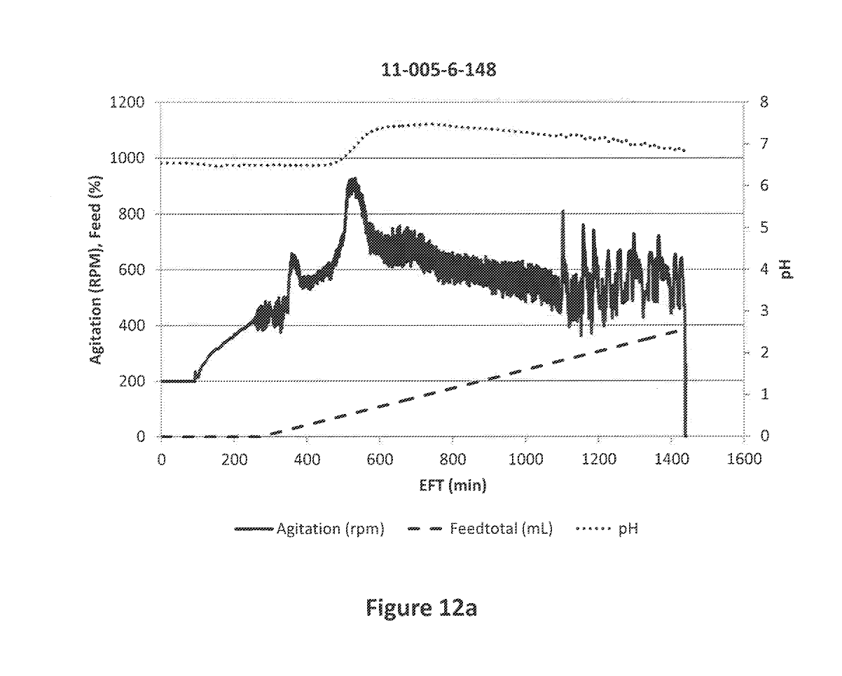

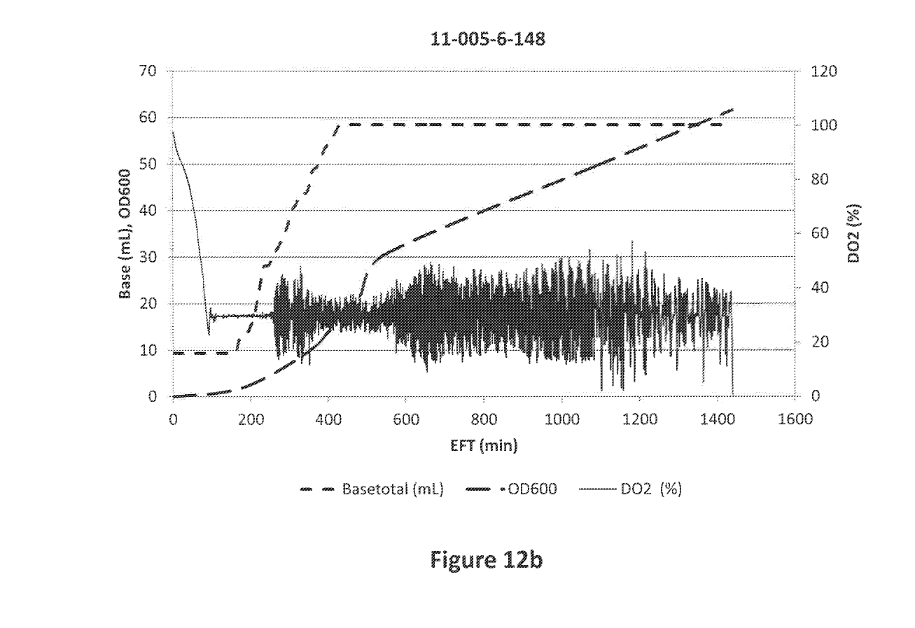

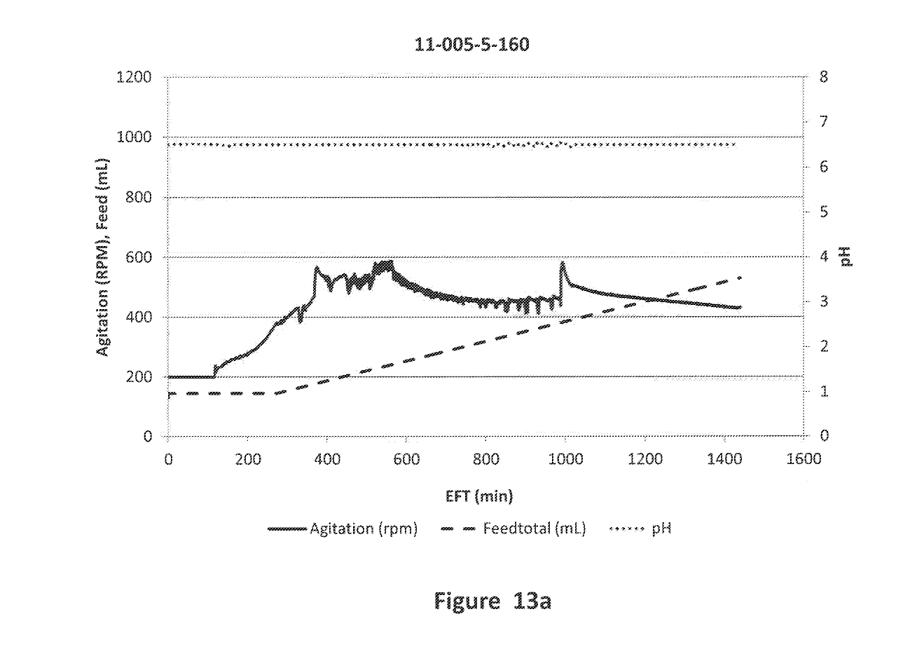

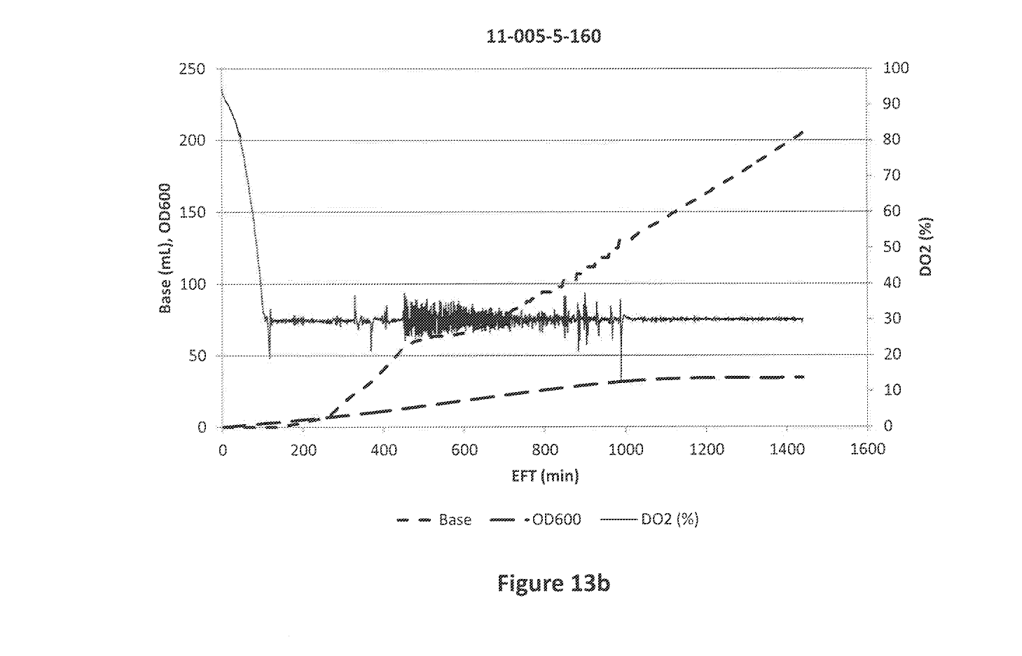

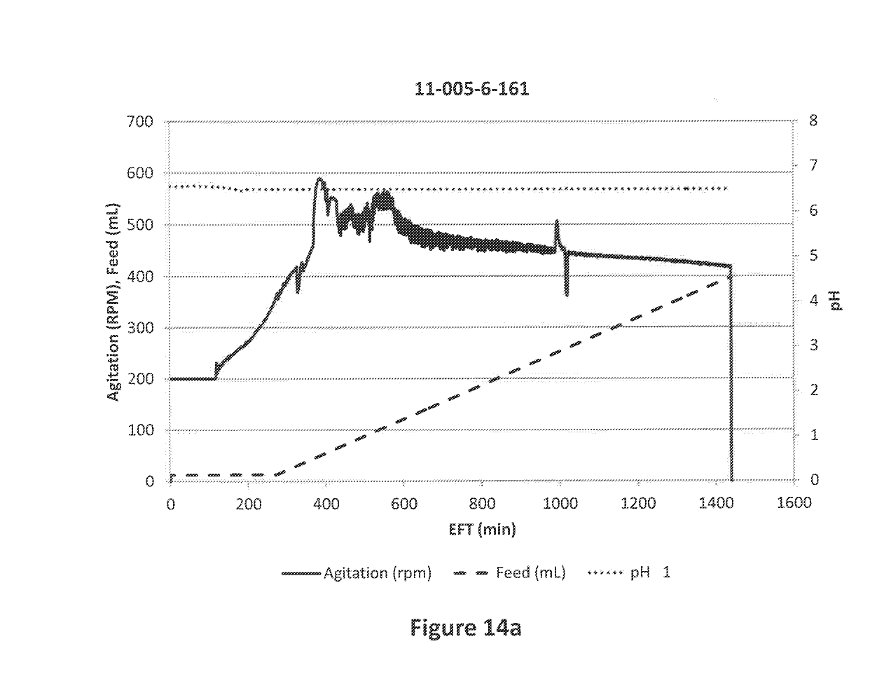

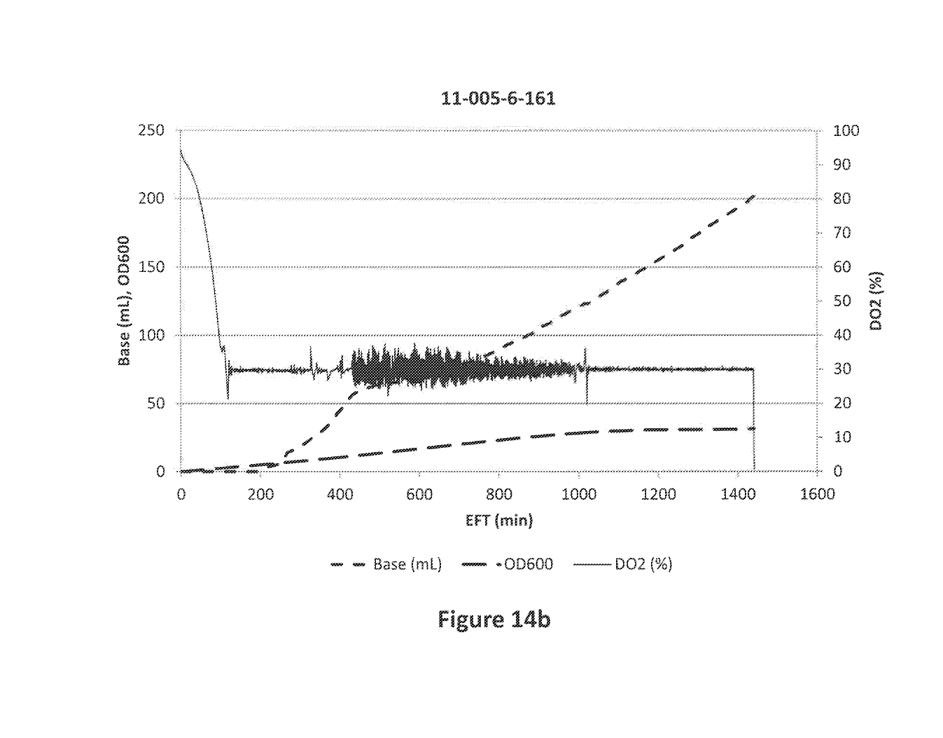

Table 19 shows the concentrations of the eight standards and FIG. 5 shows a typical standard curve.

Preparing the Standard Curve:
(1) Set up three microcentrifuge tubes in a rack, skip two spaces, set up eight more tubes. Label them 1-11.
(2) Pipette 90 μl of PBS into tube 1.
(3) Pipette 450 μl of PBS into tubes 2, 3, and 5.
(4) Pipette 400 μl of PBS into tube 4.
(5) Pipette 250 μl of PBS into tubes 6-11.
(6) Pipette 10 μl of wild type phage (production lot) into tube 1. Vortex.
(7) Pipette 50 μl from tube 1 to tube 2. Vortex.
(8) Pipette 50 μl from tube 2 to tube 3. Vortex.
(9) Pipette 100 μl from tube 3 to tube 4. Vortex.
(10) Pipette 50 μl from tube 3 to tube 5. Vortex.
(11) Pipette 250 μl from tube 5 to tube 6. Vortex.
(12) Pipette 250 μl from tube 6 to tube 7. Vortex.
(13) Pipette 250 μl from tube 7 to tube 8. Vortex.
(14) Pipette 250 μl from tube 8 to tube 9. Vortex.
(15) Pipette 250 μl from tube 9 to tube 10. Vortex.
(16) Pipette 250 μl from tube 10 to tube 11. Vortex.
(17) Pipette 100 μl into two wells of 96-well plate of tubes 4-11.
(18) Pipette 100 μl of PBS into four wells as the blank.

### Example 9

### Experimental Results from Exemplary Process 2

The following table shows the results of 5 separate experiments using the protocol described above in “Process 2.” In summary, an E. coli culture was grown to an OD₆₀₀ (density) of 1-4 in the second of two shake flask cultures grown in series. The E. coli culture was transferred into a fermentor and 2.8×10⁸ M13 phage/OD₆₀₀/mL were added to the fermentor once the OD₆₀₀ of the E. coli culture in the fermentor had reached and OD₆₀₀ of 55+/−5. The fermentor was kept at a temperature of 37° C., dissolved oxygen content of 30%, and a pH of 6.5 (controlled with ammonium hydroxide). Titers greater than 4×10¹² bacteriophage (M13) per mL were obtained in each of the 5 experiments.

TABLE 20

| Run | Batch | ELISA Titer | Run | Infection | Final |
|---|---|---|---|---|---|
| # | # | (phage/mL) | Time | OD₆₀₀ | OD₆₀₀ |
| 1 | 11-005-5-110 | 9.2 × 10¹³ | 46.25 | 45.6 | 68.2 |
| 2 | 11-005-7-112 | 1.0 × 10¹⁴ | 46.25 | 47.3 | 83.7 |
| 3 | 11-005-4-146 | 3.5 × 10¹³ | 42.25 | 54.4 | 122.9 |
| 4 | 11-005-7-149 | 2.7 × 10¹³ | 42.25 | 51.9 | 140.4 |
| 5 | 11-005-7-162 | 4.2 × 10¹³ | 42.5 | 53.1 | 96.6 |

### Example 10

### Experimental Results from Exemplary Process 3

The following table shows the results of 4 separate experiments using the protocol described above in “Process 3.” In summary, an E. coli culture was grown to an OD₆₀₀ between 1 and 4 in a shake flask. The E. coli culture was infected with 50 μl of filamentous M13 bacteriophage stock (stock at 2.8×10¹⁴ bacteriophage per mL) immediately prior to transfer to the fermentor. The fermentor was kept at a temperature of 37° C., dissolved oxygen content of 30%, and a pH of 6.5 (controlled with ammonium hydroxide). Titers greater than 4×10² bacteriophage per mL were obtained in each of the 4 experiments.

TABLE 21

| Run | Batch | ELISA Titer | Run | Final |
|---|---|---|---|---|
| # | # | (phage/mL) | Time | OD₆₀₀ |
| 1 | 11-005-5-147 | 9.2 × 10¹³ | 24 | 61.5 |
| 2 | 11-005-6-148 | 4.6 × 10¹³ | 24 | 61.7 |
| 3 | 11-005-5-160 | 1.9 × 10¹³ | 24 | 34.4 |
| 4 | 11-005-6-161 | 2.2 × 10¹³ | 24 | 31.4 |

### Example 11

### Alternate Procedure for Adding M13 Phage

M13 filamentous bacteriophage were added to fermentation cultures being grown according to Exemplary Protocol 4 when the culture OD₆₀₀ (OD) was between 45 and 60. At the feed rates described above, this OD was attained between 20-28 hours after inoculation. Several experiments were performed and M13 stocks having concentrations listed in the table below were used to infect the fermentation cultures.

The E. coli culture is infected with M13 at a rate of 1×10⁵ M13 per mL culture starting volume per unit OD or 1×10⁶ M13 per mL culture starting volume per unit OD. Thus, for a 5 L final culture volume, with a starting volume of 4 L and an infection OD of 50, 2×10¹⁰ M13 particles or 2×10¹¹ M13 particles (equivalent to 0.1 mL or 1 mL of a 2×10¹¹ phage/mL stock solution) would be used to infect the culture.

To calculate the amount of M13 stock solution to add, use the following equation: M13 to add (total phage)=1×10⁶ phage multiplied by OD600 multiplied by volume (mL), or M13 to add (mL of stock)=[1×10⁶ phage/OD600/mL multiplied by OD600 multiplied by volume (mL)] divided by phage concentration (2×10¹¹ phage/mL in the scenario according to the previous paragraph).

The nutrient feed pump is stopped temporarily, and as the dissolved oxygen spikes (greater than 40%), the agitation is stopped. The air flow is kept constant at 1-1.25 vvm (corresponding to 4-5 L/min given the 4 L culture volume) and the virus suspension is aseptically added to the fermentor. The reactor is allowed to stand without agitation for 30 minutes before restarting agitation. Once agitation had been restarted and the dissolved oxygen concentration is above 20%, the feed pump is restarted at a rate as shown in Table 16.

### Example 12

### Experimental Results from Exemplary Process 4

The following table shows the results of 7 separate experiments using the protocol described above in “Exemplary Process 4.” In summary, an E. coli culture was grown to an OD₆₀₀ (density) of 1-4 in the second of two shake flask cultures grown in series. The E. coli culture was transferred into a fermentor and 1×10⁶ M13 phage/OD₆₀₀/mL were added to the fermentor once the OD₆₀₀ of the E. coli culture in the fermentor had reached and OD₆₀₀ of 45-60. The fermentor was kept at a temperature of 37° C., dissolved oxygen content of 30%, and a pH of 6.5 (controlled with ammonium hydroxide). Titers greater than 4×10¹² bacteriophage (M13) per mL were obtained in each of the 5 experiments. Several control experiments without addition of bacteriophage were also performed (data not shown).

TABLE 22

| Run | Batch | ELISA Titer | Run | Infection | Final |
|---|---|---|---|---|---|
| # | # | (phage/mL) | Time | OD₆₀₀ | OD₆₀₀ |
| 1 | 70011_3b | 5.80 × 10¹² | 48 hr | 51.3 | 74.6 |
| 2 | 70011_4a | 1.40 × 10¹³ | 48 hr | 46.7 | 81.3 |
| 3 | 70011_4b | 1.40 × 10¹³ | 48 hr | 45.7 | 71.3 |
| 4 | 70011_5a | 2.00 × 10¹³ | 47 hr 37 min | 47.7 | 62.67 |
| 5 | 70011_5b | 1.50 × 10¹³ | 48 hr | 57.3 | 83.67 |
| 6 | 70011_6a | 1.41 × 10¹³ | 48 hr | 49.67 | 73.67 |
| 7 | 70011_6b | 1.0 × 10¹³ | 48 hr | 54.67 | 104 |

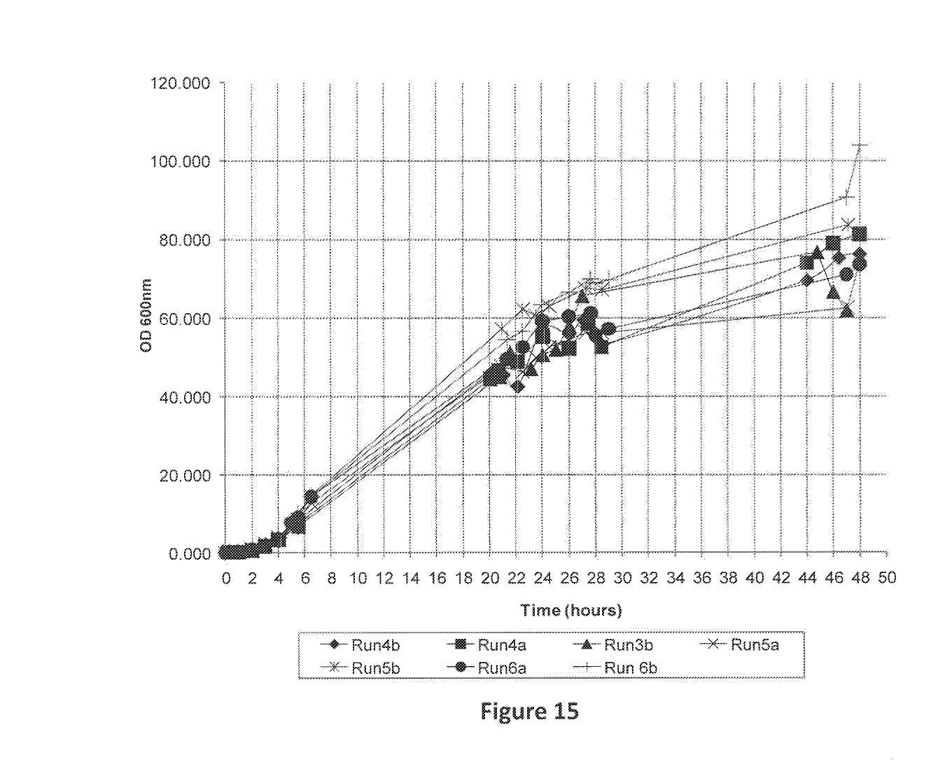

FIG. 15 shows a plot of OD600 versus time for these experiments. In FIG. 15, “Run 3b” refers to Batch 70011_{—}3b, “Run 4a” refers to Batch 70011_{—}4a, etc.

### Example 13

### Additional Experimental Results from Exemplary Process 4

Additional experiments were performed according to exemplary process 4 in which the amount of M13 phage added was 1×10⁵ phage/OD600/mL. Feed was initiated upon observation of a pH spike indicating glucose limitation, which occurred at approximately 5.5 hours. The conditions were otherwise similar to Example 12. Results are shown in Table 23.

TABLE 23

| Run | Batch | ELISA Titer | Run | Infection | Final |
|---|---|---|---|---|---|
| # | # | (phage/mL) | Time | OD₆₀₀ | OD₆₀₀ |
| 1 | 70218_04A | 1.2 × 10¹³ | 51 hr | 49.3 | 80.33 |
| 2 | 70218_04B | 5.8 × 10¹² | 51 hr | 52.3 | 74.33 |

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A culture medium comprising filamentous bacteriophage at a concentration of at least 4×10¹² filamentous bacteriophage per milliliter (mL).

2. The culture medium of claim 1, wherein the concentration is at least 1×10¹³ filamentous bacteriophage per mL.

3. The culture medium of claim 1 or 2, wherein the filamentous bacteriophage do not display an antibody or a non-filamentous bacteriophage antigen on their surface.

4. The culture medium of any one of claims 1 to 3, wherein the filamentous bacteriophage are wild type.

5. The culture medium of any one of claims 1 to 4, wherein the filamentous bacteriophage are M13.

6. The culture medium of any one of claims 1 to 5, wherein the culture medium comprises at least 2×10¹⁶ filamentous bacteriophage.

7. The culture medium of any one of claims 1 to 6, further comprising E. coli of a strain that expresses an F pilus.

8. A fermentor comprising the culture medium of any one of claims 1 to 7, wherein the culture medium in the fermentor has a volume of at least 50 mL.

9. A method of producing a culture medium comprising greater than 4×10¹² filamentous bacteriophage per mL, comprising:
a) providing in a fermentor a culture comprising E. coli of a strain that expresses an F pilus contacted with a liquid culture medium;
b) adding filamentous bacteriophage to the culture in the fermentor, wherein the addition occurs either during the provision of step (a), or after beginning incubation according to step (c);
c) incubating the culture continuously or discontinuously for a duration totaling at least 36 hours, during which:
(i) dissolved oxygen in the culture is maintained at a concentration at or above 20%;
(ii) pH in the culture is maintained at or above 6.5; and
(iii) the culture is maintained at a temperature ranging from 30° C.-39° C.;
d) providing a supplemental carbon source to the culture as a feed beginning at a time between 3 and 7 hours after initiating incubation; and
e) ending incubation after the concentration of filamentous bacteriophage in the culture reaches a concentration greater than 4×10¹² filamentous bacteriophage per mL.

10-22. (canceled)

23. The culture medium of any one of claims 1 to 7 comprising at least 200×10¹² (or 2×10¹⁴) filamentous bacteriophage.

24. The culture medium of any one of claims 1 to 7 comprising at least 50×10¹³ (or 5×10¹⁴) filamentous bacteriophage.
